SEQUENCE LISTING (33 sequences)

SEQ ID NO: 1 - DNA, 504 bp, Arabidopsis thaliana
agattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaatgc

SEQ ID NO: 2 - DNA, 367 bp, Arabidopsis thaliana
agattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccct

SEQ ID NO: 3 - DNA, 1069 bp, Arabidopsis thaliana
agtcttaatcttgtggatagaagaatcttacaaaattcttgtgaaatttagattccaaagaatatactacgaaaaaagatttagtcttcttcctattttttgtttggtcaggattacttaaagtcgtagctccatgtgagtttataaatatcatcttattctttctctttctcaacatatgtcggtacgtctttttcttttcacgtatgtcggtacgtcaaaatgttatagtgctactcttagattgttacattcatatcaacatcagaatacccaacaatactacatacatatatccaactagtcaaatactctataaaaaactaactaaacaattcaacagacaggataaaaagaaatttggtagtctattgagcattttggcttacaagtaaaagatcttgaaacatatcaacgtaaactaataatgcatatttttaccaaaacaaaaaactaataatggatatatactataacattttggcttttggccagttaacaaaaagaaaaagaaaaaaaaagtttagaaatattaaaattatgacgttaggacaaaagaagaaaatatcaaatttatagaaaacaaccactacataaatagtaggtcggccatgggtcggacaaaatagattacttaacaattaacgagcagcaaattagctttgggtatagtaacaaccaatcaatgttccctcagcttcttcttcgtgggctccatttaggccacgtggcattatcacagccttgtattgaattcaacggagatccttcaaccaatcacgagatttcgttagcgctggtagggccctctctcgctaaacacatggggtagtatctaaagtggacctgtcacactgcatcgccatgtcatcatttcgggcatcttcaacttaatacgaacttacgaagctttccccggtggataattaaccgttttattaattagccataatcacggcctcaaagcctatataagttgtttctcaccaacaatcaaatcacttatcaacaactcaaaactgatcatcacaaagaaaagagttttctctgataatctttctcggacaag

SEQ ID NO: 4 - DNA, 850 bp, Arabidopsis thaliana
tgtttggtcaggattacttaaagtcgtagctccatgtgagtttataaatatcatcttattctttctctttctcaacatatgtcggtacgtctttttcttttcacgtatgtcggtacgtcaaaatgttatagtgctactcttagattgttacattcatatcaacatcagaatacccaacaatactacatacatatatccaactagtcaaatactctataaaaaactaactaaacaattcaacagacaggataaaaagaaatttggtagtctattgagcattttggcttacaagtaaaagatcttgaaacatatcaacgtaaactaataatgcatatttttaccaaaacaaaaaactaataatggatatatactataacattttggcttttggccagttaacaaaaagaaaaagaaaaaaaaagtttagaaatattaaaattatgacgttaggacaaaagaagaaaatatcaaatttatagaaaacaaccactacataaatagtaggtcggccatgggtcggacaaaatagattacttaacaattaacgagcagcaaattagctttgggtatagtaacaaccaatcaatgttccctcagcttcttcttcgtgggctccatttaggccacgtggcattatcacagccttgtattgaattcaacggagatccttcaaccaatcacgagatttcgttagcgctggtagggccctctctcgctaaacacatggggtagtatctaaagtggacctgtcacactgcatcgccatgtcatcatttcgggcatcttcaacttaatacgaacttacgaagctttccccggtggataattaaccgttttattaattagccat

SEQ ID NO: 5 - DNA, 1003 bp, Arabidopsis thaliana
ctttattttattttttgaatgaaaatgtcttctttattcgtaattttaaactcactggtggtggatatattgttatgtccccaattcgtctggcaactctcgtatattagtgagaaaaatttgtccattatttactgcactattaccctgtgttaattttttgtattgaaattgttttttagtaattcacgtcatatagcgaatgattctttaattttaaaaattcagtcttaagtttacaaattaaataacgctactgtaaccaactctgtacgaccaacatgttcgagtttttgtatatacggccatatatgtacatattttactataaagcgaaaaaatccataaattatttaattaatatataaaggtgccattctatttccaatgtgcttaggaaaatgcagaacctcgtgctatatctctgtcgccacgtgcaaatataacaatatgaaatagaactagcaaatcttgaaatctaactcttaagactaattcaagcacatacgtagagaaagttgaccaacggttatcagcattttaacatggaccttatcaacattttaacaaagtccacaaacaaccagtcttacaatcgcattggtacaagataatcgaattcatcttccatataacaaaacctaaaccttggtgtgaaaaggagaagatatgtatgttaaaggccgcctatgcctctggtttggggtatatgattctaagattagggtttgaatattttcgttagcctgccatgagatatatttatgtgataattagagcctcttatgcattaatgcataaccgactagatcatgtggtattcagctaatcagtacacacaagacaaagtagtaaatgagtttgatgaagactgtggtctgataattcctatcaacgttaaatctgtcggggccaggcagccagcaacattttgcctaacaacgctctgaattcaattgaacctaggctatataatagcaggctaacttaactaagagttagg

SEQ ID NO: 6 - DNA, 949 bp, Arabidopsis thaliana
tttattttattttttgaatgaaaatgtcttctttattcgtaattttaaactcactggtggtggatatattgttatgtccccaattcgtctggcaactctcgtatattagtgagaaaaatttgtccattatttactgcactattaccctgtgttaattttttgtattgaaattgttttttagtaattcacgtcatatagcgaatgattctttaattttaaaaattcagtcttaagtttacaaattaaataacgctactgtaaccaactctgtacgaccaacatgttcgagtttttgtatatacggccatatatgtacatattttactataaagcgaaaaaatccataaattatttaattaatatataaaggtgccattctatttccaatgtgcttaggaaaatgcagaacctcgtgctatatctctgtcgccacgtgcaaatataacaatatgaaatagaactagcaaatcttgaaatctaactcttaagactaattcaagcacatacgtagagaaagttgaccaacggttatcagcattttaacatggaccttatcaacattttaacaaagtccacaaacaaccagtcttacaatcgcattggtacaagataatcgaattcatcttccatataacaaaacctaaaccttggtgtgaaaaggagaagatatgtatgttaaaggccgcctatgcctctggtttggggtatatgattctaagattagggtttgaatattttcgttagcctgccatgagatatatttatgtgataattagagcctcttatgcattaatgcataaccgactagatcatgtggtattcagctaatcagtacacacaagacaaagtagtaaatgagtttgatgaagactgtggtctgataattcctatcaacgttaaatctgtcggggccaggcagccagcaacattttgcctaacaacgctctga

SEQ ID NO: 7 - DNA, 1195 bp, Glycine max
atctaggcttttgaccttgacttggtagaacctcttgccggtttgatttgttcccatgcttactaaagtgagataaaagctggtgcaaatcaaaactccgatatctcatgggtagaatggatgaatgcatgatggaatgcatatgacacagatgcaatctaggaatgcaggggtccggggaatttgtccccttcttagatacgacgtctaggggtagcgaaatgccccaacgcacgtttttaagaaggcgacacggaccctccgttggtttgtttacagtagggatcaagacagaacccatatgcaatgcctatgcaaaagacacaatgcgggaatgtgcacagtatgacaatatttaccgaacataagcaaaagggtatatgatactcatgcatggcagtgtgaaaaatggcacgcagcgtgtttgcttcgtgcccctatttaagggacctataagggagagaactgactaggcttttagtaataatccccaaggtagttatatctctcttgatggtttctagaggtatcatcacttttgaaatgaacgtataataacatgaatatatgatggcagcaaaggggccagccagtcaccacttcttccttctgatatctcttttttgttttgtttctttcttttttatttataaaaatactctttgtgtataggaatactattttttaagatacaaacagaaatatatttttagaatattatcttaataaatacatttttagcatttattttattaaattttatcttattctgtaaaaaaatgtcttatttattaatgtatgtttcagtaaatcttatcttttttttttacgtctcaataaatcttattatcttatttggatacatatttttgtcaaaatatttatataaaaaataaatttgaaaaaaagttaggtggcttattgggctggatgaaagagagtcaggcccgccacgggcccacggtttggaaaattcagatctgcgagcgtgaaaattatgcatacccaactagcaacgccaacgactctaaagaacattctggaaggactccaaaccctaactattctactatataagctaactattgcccctcagatctctcacttgaatcacagtggtgccttatcgctgtggttttcatcgtcttccttctcttatatttcttttttttttctctacccgcacgccgaaacc

SEQ ID NO: 8 - DNA, 1132 bp, Glycine max
aggcttttgaccttgacttggtagaacctcttgccggtttgatttgttcccatgcttactaaagtgagataaaagctggtgcaaatcaaaactccgatatctcatgggtagaatggatgaatgcatgatggaatgcatatgacacagatgcaatctaggaatgcaggggtccggggaatttgtccccttcttagatacgacgtctaggggtagcgaaatgccccaacgcacgtttttaagaaggcgacacggaccctccgttggtttgtttacagtagggatcaagacagaacccatatgcaatgcctatgcaaaagacacaatgcgggaatgtgcacagtatgacaatatttaccgaacataagcaaaagggtatatgatactcatgcatggcagtgtgaaaaatggcacgcagcgtgtttgcttcgtgcccctatttaagggacctataagggagagaactgactaggcttttagtaataatccccaaggtagttatatctctcttgatggtttctagaggtatcatcacttttgaaatgaacgtataataacatgaatatatgatggcagcaaaggggccagccagtcaccacttcttccttctgatatctcttttttgttttgtttctttcttttttatttataaaaatactctttgtgtataggaatactattttttaagatacaaacagaaatatatttttagaatattatcttaataaatacatttttagcatttattttattaaattttatcttattctgtaaaaaaatgtcttatttattaatgtatgtttcagtaaatcttatcttttttttttacgtctcaataaatcttattatcttatttggatacatatttttgtcaaaatatttatataaaaaataaatttgaaaaaaagttaggtggcttattgggctggatgaaagagagtcaggcccgccacgggcccacggtttggaaaattcagatctgcgagcgtgaaaattatgcatacccaactagcaacgccaacgactctaaagaacattctggaaggactccaaaccctaactattctactatataagctaactattgcccctcagatctctcacttgaatcacagtggtgccttatcgctgtggt

SEQ ID NO: 9 - DNA, 47 bp, Cauliflower mosaic virus
tcgcaagacccttcctctatataaggaagttcatttcatttggagag

SEQ ID NO: 10 - DNA, 79 bp, Oryza sativa
gtccctccgcttccaaagaaacgccccccatcgccactatatacatacccccccctctcctcccatccccccaacccta

SEQ ID NO: 11 - DNA, 461 bp, Artificial sequence
  Synthetic sequence
attttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttaggtaccatggcatgcatcgatagatctcgaggagcttcgcaagacccttcctctatataaggaagttcatttcatttggagaggacacgctga

SEQ ID NO: 12 - DNA, 480 bp, Artificial sequence
  Synthetic sequence
attttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttaggtaccatggcatgcatcgatagatctcgaggtccctccgcttccaaagaaacgccccccatcgccactatatacatacccccccctctcctcccatccccccaaccctacc

SEQ ID NO: 13 - DNA, 918 bp, Artificial sequence
  Synthetic sequence
tgtttggtcaggattacttaaagtcgtagctccatgtgagtttataaatatcatcttattctttctctttctcaacatatgtcggtacgtctttttcttttcacgtatgtcggtacgtcaaaatgttatagtgctactcttagattgttacattcatatcaacatcagaatacccaacaatactacatacatatatccaactagtcaaatactctataaaaaactaactaaacaattcaacagacaggataaaaagaaatttggtagtctattgagcattttggcttacaagtaaaagatcttgaaacatatcaacgtaaactaataatgcatatttttaccaaaacaaaaaactaataatggatatatactataacattttggcttttggccagttaacaaaaagaaaaagaaaaaaaaagtttagaaatattaaaattatgacgttaggacaaaagaagaaaatatcaaatttatagaaaacaaccactacataaatagtaggtcggccatgggtcggacaaaatagattacttaacaattaacgagcagcaaattagctttgggtatagtaacaaccaatcaatgttccctcagcttcttcttcgtgggctccatttaggccacgtggcattatcacagccttgtattgaattcaacggagatccttcaaccaatcacgagatttcgttagcgctggtagggccctctctcgctaaacacatggggtagtatctaaagtggacctgtcacactgcatcgccatgtcatcatttcgggcatcttcaacttaatacgaacttacgaagctttccccggtggataattaaccgttttattaattagccatctcgaggagcttcgcaagacccttcctctatataaggaagttcatttcatttggagaggacacgctga

SEQ ID NO: 14 - DNA, 1033 bp, Artificial sequence
  Synthetic sequence
tttattttattttttgaatgaaaatgtcttctttattcgtaattttaaactcactggtggtggatatattgttatgtccccaattcgtctggcaactctcgtatattagtgagaaaaatttgtccattatttactgcactattaccctgtgttaattttttgtattgaaattgttttttagtaattcacgtcatatagcgaatgattctttaattttaaaaattcagtcttaagtttacaaattaaataacgctactgtaaccaactctgtacgaccaacatgttcgagtttttgtatatacggccatatatgtacatattttactataaagcgaaaaaatccataaattatttaattaatatataaaggtgccattctatttccaatgtgcttaggaaaatgcagaacctcgtgctatatctctgtcgccacgtgcaaatataacaatatgaaatagaactagcaaatcttgaaatctaactcttaagactaattcaagcacatacgtagagaaagttgaccaacggttatcagcattttaacatggaccttatcaacattttaacaaagtccacaaacaaccagtcttacaatcgcattggtacaagataatcgaattcatcttccatataacaaaacctaaaccttggtgtgaaaaggagaagatatgtatgttaaaggccgcctatgcctctggtttggggtatatgattctaagattagggtttgaatattttcgttagcctgccatgagatatatttatgtgataattagagcctcttatgcattaatgcataaccgactagatcatgtggtattcagctaatcagtacacacaagacaaagtagtaaatgagtttgatgaagactgtggtctgataattcctatcaacgttaaatctgtcggggccaggcagccagcaacattttgcctaacaacgctctgactcgaggagcttcgcaagacccttcctctatataaggaagttcatttcatttggagaggacacgctgacaagctgactctagca

SEQ ID NO: 15 - DNA, 1036 bp, Artificial sequence
  Synthetic sequence
tttattttattttttgaatgaaaatgtcttctttattcgtaattttaaactcactggtggtggatatattgttatgtccccaattcgtctggcaactctcgtatattagtgagaaaaatttgtccattatttactgcactattaccctgtgttaattttttgtattgaaattgttttttagtaattcacgtcatatagcgaatgattctttaattttaaaaattcagtcttaagtttacaaattaaataacgctactgtaaccaactctgtacgaccaacatgttcgagtttttgtatatacggccatatatgtacatattttactataaagcgaaaaaatccataaattatttaattaatatataaaggtgccattctatttccaatgtgcttaggaaaatgcagaacctcgtgctatatctctgtcgccacgtgcaaatataacaatatgaaatagaactagcaaatcttgaaatctaactcttaagactaattcaagcacatacgtagagaaagttgaccaacggttatcagcattttaacatggaccttatcaacattttaacaaagtccacaaacaaccagtcttacaatcgcattggtacaagataatcgaattcatcttccatataacaaaacctaaaccttggtgtgaaaaggagaagatatgtatgttaaaggccgcctatgcctctggtttggggtatatgattctaagattagggtttgaatattttcgttagcctgccatgagatatatttatgtgataattagagcctcttatgcattaatgcataaccgactagatcatgtggtattcagctaatcagtacacacaagacaaagtagtaaatgagtttgatgaagactgtggtctgataattcctatcaacgttaaatctgtcggggccaggcagccagcaacattttgcctaacaacgctctgactcgaggtccctccgcttccaaagaaacgccccccatcgccactatatacatacccccccctctcctcccatccccccaaccctacc

SEQ ID NO: 16 - DNA, 1216 bp, Artificial sequence
  Synthetic sequence
aggcttttgaccttgacttggtagaacctcttgccggtttgatttgttcccatgcttactaaagtgagataaaagctggtgcaaatcaaaactccgatatctcatgggtagaatggatgaatgcatgatggaatgcatatgacacagatgcaatctaggaatgcaggggtccggggaatttgtccccttcttagatacgacgtctaggggtagcgaaatgccccaacgcacgtttttaagaaggcgacacggaccctccgttggtttgtttacagtagggatcaagacagaacccatatgcaatgcctatgcaaaagacacaatgcgggaatgtgcacagtatgacaatatttaccgaacataagcaaaagggtatatgatactcatgcatggcagtgtgaaaaatggcacgcagcgtgtttgcttcgtgcccctatttaagggacctataagggagagaactgactaggcttttagtaataatccccaaggtagttatatctctcttgatggtttctagaggtatcatcacttttgaaatgaacgtataataacatgaatatatgatggcagcaaaggggccagccagtcaccacttcttccttctgatatctcttttttgttttgtttctttcttttttatttataaaaatactctttgtgtataggaatactattttttaagatacaaacagaaatatatttttagaatattatcttaataaatacatttttagcatttattttattaaattttatcttattctgtaaaaaaatgtcttatttattaatgtatgtttcagtaaatcttatcttttttttttacgtctcaataaatcttattatcttatttggatacatatttttgtcaaaatatttatataaaaaataaatttgaaaaaaagttaggtggcttattgggctggatgaaagagagtcaggcccgccacgggcccacggtttggaaaattcagatctgcgagcgtgaaaattatgcatacccaactagcaacgccaacgactctaaagaacattctggaaggactccaaaccctaactattctactatataagctaactattgcccctcagatctctcacttgaatcacagtggtgccttatcgctgtggtctcgaggagcttcgcaagacccttcctctatataaggaagttcatttcatttggagaggacacgctgacaagctgactctagca

SEQ ID NO: 17 - DNA, 1219 bp, Artificial sequence
  Synthetic sequence
aggcttttgaccttgacttggtagaacctcttgccggtttgatttgttcccatgcttactaaagtgagataaaagctggtgcaaatcaaaactccgatatctcatgggtagaatggatgaatgcatgatggaatgcatatgacacagatgcaatctaggaatgcaggggtccggggaatttgtccccttcttagatacgacgtctaggggtagcgaaatgccccaacgcacgtttttaagaaggcgacacggaccctccgttggtttgtttacagtagggatcaagacagaacccatatgcaatgcctatgcaaaagacacaatgcgggaatgtgcacagtatgacaatatttaccgaacataagcaaaagggtatatgatactcatgcatggcagtgtgaaaaatggcacgcagcgtgtttgcttcgtgcccctatttaagggacctataagggagagaactgactaggcttttagtaataatccccaaggtagttatatctctcttgatggtttctagaggtatcatcacttttgaaatgaacgtataataacatgaatatatgatggcagcaaaggggccagccagtcaccacttcttccttctgatatctcttttttgttttgtttctttcttttttatttataaaaatactctttgtgtataggaatactattttttaagatacaaacagaaatatatttttagaatattatcttaataaatacatttttagcatttattttattaaattttatcttattctgtaaaaaaatgtcttatttattaatgtatgtttcagtaaatcttatcttttttttttacgtctcaataaatcttattatcttatttggatacatatttttgtcaaaatatttatataaaaaataaatttgaaaaaaagttaggtggcttattgggctggatgaaagagagtcaggcccgccacgggcccacggtttggaaaattcagatctgcgagcgtgaaaattatgcatacccaactagcaacgccaacgactctaaagaacattctggaaggactccaaaccctaactattctactatataagctaactattgcccctcagatctctcacttgaatcacagtggtgccttatcgctgtggtctcgaggtccctccgcttccaaagaaacgccccccatcgccactatatacatacccccccctctcctcccatccccccaaccctacc

SEQ ID NO: 18 - DNA, 26 bp, Artificial sequence
  Synthetic primer
ggatccagattttacgtataaaataa

SEQ ID NO: 19 - DNA, 25 bp, Artificial sequence
  Synthetic primer
aggcctccaatagaagtaatcaaac

SEQ ID NO: 20 - DNA, 26 bp, Artificial Sequence
  Synthetic primer
ggatccagtcttaatcttgtggatag

SEQ ID NO: 21 - DNA, 24 bp, Artificial Sequence
  Synthetic primer
aggccttgtccgagaaagattatc

SEQ ID NO: 22 - DNA, 26 bp, Artificial Sequence
  Synthetic primer
ctcgagtgtttggtcaggattactta

SEQ ID NO: 23 - DNA, 26 bp, Artificial Sequence
  Synthetic primer
ctcgagatggctaattaataaaacgg

SEQ ID NO: 24 - DNA, 27 bp, Artificial Sequence
  Synthetic primer
ggtaccagattttacgtataaaataaa

SEQ ID NO: 25 - DNA, 26 bp, Artificial Sequence
  Synthetic primer
ggtacctaaagggacacgtatgaagc

SEQ ID NO: 26 - DNA, 80 bp, Artificial Sequence
  Synthetic primer
tccctccgcttccaaagaaacgccccccatcgccactatatacatacccccccctctcctcccatccccccaaccctacc

SEQ ID NO: 27 - DNA, 80 bp, Artificial Sequence
  Synthetic primer
ggtagggttggggggatgggaggagaggggggggtatgtatatagtggcgatggggggcgtttctttggaagcggaggga

SEQ ID NO: 28 - DNA, 26 bp, Artificial Sequence
  Synthetic primer
aaggatcctttattttattttttgaa

SEQ ID NO: 29 - DNA, 26 bp, Artificial Sequence
  Synthetic primer
ttaggcctaactcttagttaagttag

SEQ ID NO: 30 - DNA, 26 bp, Artificial Sequence
  Synthetic primer
aactcgaggcttttgaccttgacttg

SEQ ID NO: 31 - DNA, 27 bp, Artificial Sequence
  Synthetic primer
ttctcgagaccacagcgataaggcacc

SEQ ID NO: 32 - DNA, 24 bp, Artificial Sequence
  Synthetic primer
ctcgagtttattttattttttgaa

SEQ ID NO: 33 - DNA, 26 bp, Artificial Sequence
  Synthetic primer
ctcgagtcagagcgttgttaggcaaa

(19) United States
(12) Patent Application Publication
(10) Pub. No.: US 2010/0275326 A1
(43) Pub. Date: Oct. 28, 2010

(54) ENGINEERED DICOTYLEDONOUS PROMOTERS CAPABLE OF EXPRESSING IN MONOCOTYLEDONOUS PLANTS

(76) Inventors: Santanu Dasgupta, Maharashtra (IN); Targolli L. Jayaprakash, Karnataka (IN)

Correspondence Address: SONNENSCHEIN NATH & ROSENTHAL LLP, P.O. BOX 061080, SOUTH WACKER DRIVE STATION, WILLIS TOWER, CHICAGO, IL, 60606, US

(21) Appl. No.: 12/676,601

(22) PCT Filed: Oct. 8, 2008
(86) PCT No.: PCT/US08/79223

§ 371 Date: Jun. 30, 2010

(30) Foreign Application Priority Data
Oct. 8, 2007 (IN) 2106/DEL/2007

(51) Int. Cl. A01H 1/00; C07H 21/00; C12N 15/79; C12N 5/10; A01H 5/10; A01H 5/00

(52) U.S. Cl. 800/278; 536/24.1; 435/320.1; 435/419; 800/295; 800/298

(57) ABSTRACT

The present invention relates to an engineered plant expression element. More specifically the present invention provides polynucleotide molecules and constructs, wherein said polynucleotide molecules comprise a promoter from a dicotyledonous species in which the native dicotyledonous TATA box to TSS region is substituted with a TATA box to TSS region of a promoter from a viral or monocotyledonous source, resulting in a chimeric promoter, or promoter element, which when operably linked to a transcribable polynucleotide molecule expresses said transcribable polynucleotide molecule in a monocotyledonous plant. The present invention also relates to a vector, a cell, and a transgenic plant containing the chimeric promoter operably linked to a transcribable molecule, including a reporter gene or a gene of agronomic interest.

This application claims priority to Indian Application No. 2106/DELNP/2007, filed Oct. 8, 2007, which is incorporated herein by reference in its entirety.

### INCORPORATION OF SEQUENCE LISTING

A sequence listing containing the file named MONS178WO_ST25.txt, which is 24,896 bytes (as measured in Microsoft Windows®) and created on Oct. 8, 2008, comprises 33 nucleotide sequences, and is herein incorporated by reference in its entirety.

### FIELD OF THE INVENTION

The present invention relates to the field of plant molecular biology and plant genetic engineering and polynucleotide molecules useful for gene expression in plants. Specifically, the present invention discloses engineered nucleic acid sequences comprising gene expression regulatory elements, such as promoters. The invention further discloses methods of producing and using said regulatory elements.

### BACKGROUND

One of the goals of plant genetic engineering is to produce plants with agronomically desirable characteristics or traits. The proper expression of a desirable transgene in a transgenic plant is one way to achieve this goal. Elements having gene regulatory activity, i.e. regulatory elements such as promoters, leaders, introns and transcription termination regions, are polynucleotide molecules which play an integral part in the overall expression of genes in living cells. Isolated regulatory elements that function in plants are therefore useful for modifying plant phenotypes through the methods of genetic engineering.

Many regulatory elements are available and are useful for providing an appropriate level of gene expression. For example, constitutive promoters such as P-FMV, the promoter from the 35S transcript of the Figwort mosaic virus (U.S. Pat. No. 6,051,753, herein incorporated by reference); P-CaMV 35S, the promoter from the 35S RNA transcript of the Cauliflower mosaic virus (U.S. Pat. No. 5,530,196, herein incorporated by reference); P-Corn Actin 1, the promoter from the actin 1 gene of Oryza sativa (U.S. Pat. No. 5,641,876, herein incorporated by reference); and P-NOS, the promoter from the nopaline synthase gene of Agrobacterium tumefaciens are known to provide some level of gene expression in most or all tissues of a plant during most or all of the plant's lifespan. While previous work has provided a number of regulatory elements useful to affect gene expression in transgenic plants, there is still a great need for novel regulatory elements with beneficial expression characteristics. Many previously identified regulatory elements fail to provide the patterns or levels of expression required to fully realize the benefits of expression of selected genes in transgenic crop plants. One example of this is the need for regulatory elements capable of driving gene expression in different types of tissues. Another example is the need for elements other than promoters to provide alternate mechanisms for the regulation of gene expression. Yet another example is the need for having a promoters that can express both in monocotyledonous and dicotyledonous plants.

The genetic enhancement of plants and seeds provides significant benefits to society. For example, plants and seeds may be enhanced to have desirable agricultural, biosynthetic, commercial, chemical, insecticidal, industrial, nutritional, or pharmaceutical properties. Despite the availability of many molecular tools, however, the genetic modification of plants and seeds is often constrained by an insufficient or poorly localized expression of the engineered transgene.

Many intracellular processes may impact overall transgene expression, including transcription, translation, protein assembly and folding, methylation, phosphorylation, transport, and proteolysis. Intervention in one or more of these processes can increase the amount of transgene expression in genetically engineered plants and seeds. For example, raising the steady-state level of mRNA in the cytosol often yields an increased accumulation of transgene expression. Many factors may contribute to increasing the steady-state level of an mRNA in the cytosol, including the rate of transcription, promoter strength and other regulatory features of the promoter, efficiency of mRNA processing, and the overall stability of the mRNA.

Efforts to engineer promoters to enhance or expand expression patterns include designing chimeric promoters comprising elements from similar sources, and designing chimeric promoter-intron systems from different sources (as described in US Patent Application Publication 20070204367 to Flasinski et al. herein incorporated by reference in its entirety).

Many dicot promoters are not active in monocot system such as rice or corn. The reason for inactivity of dicot promoters in a monocot system is not clearly understood.

It is of immense social, ecological and economic interests to develop plants that have enhanced nutrition, improved resistance to pests, and tolerance to harsh conditions such as drought. Thus, the identification of new genes, regulatory elements (e.g., promoters), etc. that function in various types of plants is useful in developing enhanced varieties of crops. Clearly, there exists a need in the art for new regulatory elements, such as promoters, that are capable of expressing heterologous nucleic acid sequences in important crop species.

### SUMMARY

The present invention provides a regulatory polynucleotide molecule wherein said polynucleotide molecule comprises a promoter from a dicotyledonous gene, or a complement thereof, in which the native portion of the promoter nucleotide sequence from the TATA box to the transcription start site is substituted with the sequence from the TATA box to the transcription start site of another promoter selected from the group consisting of a plant virus promoter and a monocotyledonous promoter.

In one embodiment, the present invention provides a regulatory polynucleotide molecule wherein said polynucleotide molecule comprises a promoter from a dicotyledonous gene, or a complement thereof, selected from the group consisting of AtRd29A, Gm571, At.GolS3 and AtYP0104, in which the native portion of the promoter from the TATA box to the transcription start site is substituted with the TATA box to the transcription start site of another promoter selected from the group consisting of a plant virus promoter and a monocotyledonous promoter.

In another embodiment, the present invention provides a regulatory polynucleotide molecule wherein said polynucleotide molecule comprises a promoter from a dicotyledonous gene, or a complement thereof, in which the native portion of the promoter from the TATA box to the transcription start site is substituted with the TATA box to the transcription start site of another promoter selected from the group consisting of the 35S promoter from CaMV and a promoter from the Rice Actin1 gene.

In yet another embodiment, the present invention provides a plant cell transformed to contain a the polynucleotide construct containing a regulatory polynucleotide molecule wherein said polynucleotide molecule comprises a promoter from a dicotyledonous gene, or a complement thereof, in which the native portion of the promoter from the TATA box to the transcription start site is substituted with the TATA box to the transcription start site of another promoter selected from the group consisting of a plant virus promoter and a monocotyledonous promoter.

In still yet another embodiment, the present invention provides a plant transformed to contain a the polynucleotide construct containing a regulatory polynucleotide molecule wherein said polynucleotide molecule comprises a promoter from a dicotyledonous gene, or a complement thereof, in which the native portion of the promoter from the TATA box to the transcription start site is substituted with the TATA box to the transcription start site of another promoter selected from the group consisting of a plant virus promoter and a monocotyledonous promoter.

Additionally, the present invention provides a method of improving the expression of a dicot promoter in a monocot plant comprising substituting the native portion of the promoter from the TATA box to the transcription start site with the TATA box to the transcription start site from a promoter selected from the group consisting of a plant virus promoter and a monocot promoter.

Additional aspects of the invention will become apparent from the specification and the invention is not intended to be limited except as provided in the claims.

### DETAILED DESCRIPTION OF THE INVENTION

The invention provides polynucleotide molecules having gene regulatory activity. The design, construction, and use of these polynucleotide molecules are one object of this invention. These polynucleotide molecules are capable of affecting the expression of an operably linked transcribable polynucleotide molecule in plant tissues and can selectively regulate gene expression in transgenic plants. The present invention also provides methods of modifying, producing, and using the same. The invention also includes compositions, transformed host cells, transgenic plants, and seeds containing the promoters, and methods for preparing and using the same.

### Polynucleotide Molecules

Many types of regulatory sequences control gene expression. Not all genes are turned on at all times during the life cycle of a plant. Different genes are required for the completion of different steps in the developmental and sexual maturation of the plant. Two general types of control can be described: temporal regulation, in which a gene is only expressed at a specific time in development (for example, during flowering), and spatial regulation, in which a gene is only expressed in a specific location in the plant (for example, seed storage proteins). Many genes, however, may fall into both classes. For example, seed storage proteins are only expressed in the seed, but they also are only expressed during a short period of time during the development of the seed. Furthermore, because the binding of RNA Polymerase II to the promoter is the key step in gene expression, it follows that sequences may exist in the promoter that control temporal and spatial gene expression.

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “polynucleotide molecule” refers to the single- or double-stranded DNA or RNA molecule of genomic or synthetic origin, i.e., a polymer of deoxyribonucleotide or ribonucleotide bases, respectively, read from the 5′ (upstream) end to the 3′ (downstream) end.

As used herein, the term “polynucleotide sequence” refers to the sequence of a polynucleotide molecule. The nomenclature for nucleotide bases as set forth at 37 CFR §1.822 is used herein.

As used herein, the term “transcribable polynucleotide molecule” refers to any polynucleotide molecule capable of being transcribed into a RNA molecule, including but not limited to protein coding sequences (e.g. transgenes) and molecules useful for gene suppression.

The phrases “coding sequence” and “structural sequence” refer to a physical structure comprising an orderly arrangement of nucleic acids. In one embodiment, the nucleic acids can be arranged in a series of nucleic acid triplets that each form a codon. Each codon encodes for a specific amino acid. In this embodiment, the coding sequence, structural sequence, and transcribable polynucleotide sequence encode a series of amino acids forming a protein, polypeptide, or peptide sequence. The coding sequence, structural sequence, and transcribable polynucleotide sequence may be contained, without limitation, within a larger nucleic acid molecule, vector, etc. In addition, the orderly arrangement of nucleic acids in these sequences may be depicted, without limitation, in the form of a sequence listing, figure, table, electronic medium, etc.

As used herein, the term “regulatory element” refers to a polynucleotide molecule that has the ability to affect the transcription or translation of an operably linked transcribable polynucleotide molecule. Regulatory elements such as promoters, leaders, introns, and transcription termination regions are included in the term polynucleotide molecules and can have gene regulatory activity which can play an integral part in the overall expression of genes in living cells. Isolated regulatory elements that function in plants are useful for modifying plant phenotypes through the methods of genetic engineering. In particular embodiments, regulatory elements determine if, when, and at what level a particular gene is expressed. Regulatory polynucleotide sequences specifically interact with regulatory proteins or other proteins.

The term “native” is used to describe the environment in which a particular molecule or sequence is naturally found, i.e., a promoter associated with its naturally-associated gene, i.e. a non-heterologous relationship. For example, a rice actin 1 promoter is in nature associated with a rice actin 1 gene, which may be described as its native environment. As a further example, a rice actin 1 promoter associated with a GUS gene would be in a heterologous, or non-native, environment.

As used herein, the term “chimeric” refers to a polynucleotide molecule that is created from two or more sources, i.e. a first molecule from one gene or organism and a second molecule from another gene or organism. By the term “chimeric”, it is intended that the referenced polynucleotide molecule comprises a polynucleotide sequence that does not naturally occur.

As used herein, the term “engineered” refers to the method of creating a polynucleotide molecule that does not naturally occur.

As used herein, the term “operably linked” refers to a first polynucleotide molecule, such as a promoter, connected with a second polynucleotide molecule, which can be transcribable, such as a gene of interest. In general, operably linked refers to a polynucleotide molecule arranged so that it affects the function of another polynucleotide molecule. The two polynucleotide molecules may be part of a single contiguous polynucleotide molecule and may be adjacent. For example, a promoter is operably linked to a gene of interest if the promoter modulates transcription of the gene of interest in a cell.

As used herein, the term “gene regulatory activity” refers to a polynucleotide molecule capable of affecting transcription or translation of an operably linked polynucleotide molecule. An isolated polynucleotide molecule having gene regulatory activity may provide temporal or spatial expression or modulate levels and rates of expression of the operably linked polynucleotide molecule. An isolated polynucleotide molecule having gene regulatory activity may comprise a promoter, intron, leader, or 3′ transcriptional termination region.

As used herein, the term “gene expression” or “expression” refers to the transcription of a DNA molecule into a transcribed RNA molecule. Gene expression may be described as related to temporal, spatial, developmental, or morphological qualities as well as quantitative or qualitative indications. The transcribed RNA molecule may be translated to produce a protein molecule or may provide an antisense or other regulatory RNA molecule.

As used herein, an “expression pattern” is any pattern of differential gene expression. In particular embodiments, an expression pattern is selected from the group consisting of tissue, temporal, spatial, developmental, stress, environmental, physiological, pathological, cell cycle, and chemically responsive expression patterns.

As used herein, an “enhanced expression pattern” is any expression pattern for which an operably linked nucleic acid sequence is expressed at a level greater than 0.01%; such as in a range of about 0.5% to about 20% (w/w), of the total cellular RNA or protein.

The present invention includes a regulatory polynucleotide molecule. Preferably, the present invention includes a polynucleotide molecule which comprises a promoter from a dicotyledonous gene, or a complement thereof, in which the native portion of the promoter from the TATA box to the transcription start site is substituted with the TATA box to the transcription start site of another promoter selected from the group consisting of a plant virus promoter and a monocotyledonous promoter.

Any dicot promoter can be used in the scope of the present invention. Promoters with function in dicot plants are all well taught in the art. The present invention can be used with constitutive promoters, inducible promoters and tissue specific promoters

Many examples of plant inducible promoters are known in the art. Useful inducible promoters include, without limitation, promoters induced by salicylic acid or polyacrylic acids (PR-1; Williams, et al., Biotechnology 10:540-543, 1992); those induced by application of safeners (substituted benzenesulfonamide herbicides; Hershey and Stoner, Plant Mol. Biol. 17: 679-690, 1991); heat-shock promoters (e.g. Ou-Lee et al., Proc. Natl. Acad. Sci. U.S.A. 83: 6815, 1986; Ainley et al., Plant Mol. Biol. 14: 949, 1990); a nitrate-inducible promoter derived from the spinach nitrite reductase transcribable polynucleotide sequence (Back et al., Plant Mol. Biol. 17: 9, 1991); hormone-inducible promoters (Yamaguchi-Shinozaki et al., Plant Mol. Biol. 15: 905, 1990); and light-inducible promoters associated with the small subunit of RuBP carboxylase and LHCP families (e.g. Kuhlemeier et al., Plant Cell 1: 471, 1989; Feinbaum et al., Mol. Gen. Genet. 226: 449-456, 1991; Weisshaar, et al., EMBO J. 10: 1777-1786, 1991; Lam and Chua, J. Biol. Chem. 266: 17131-17135, 1990; Castresana et al., EMBO J. 7: 1929-1936, 1988; Schulze-Lefert, et al., EMBO J. 8: 651, 1989).

Examples of useful tissue-specific, developmentally-regulated promoters include, without limitation, the β-conglycinin 7Sα promoter (Doyle et al., J. Biol. Chem. 261: 9228-9238, 1986; Slighton and Beachy, Planta 172: 356, 1987); and seed-specific promoters (e.g. Knutzon, et al., Proc. Natl. Acad. Sci. U.S.A. 89: 2624-2628, 1992; Bustos, et al., EMBO J. 10: 1469-1479, 1991; Lam and Chua, Science 248: 471, 1991). Plant functional promoters useful for preferential expression in a seed plastid include those from plant storage proteins and from proteins involved in fatty acid biosynthesis in oilseeds. Examples of such promoters include the 5′ regulatory regions from such transcribable polynucleotide sequences as napin (Kridl et al., Seed Sci. Res. 1: 209, 1991), phaseolin, zein, soybean trypsin inhibitor, ACP, stearoyl-ACP desaturase, and oleosin. Seed-specific regulation is discussed in EP 0 255 378.

Another exemplary tissue-specific promoter is the lectin promoter, which is specific for seed tissue. The Lectin protein in soybean seeds is encoded by a single transcribable polynucleotide sequence (Le1) that is only expressed during seed maturation and accounts for about 2 to about 5% of total seed mRNA. The lectin transcribable polynucleotide sequence and seed-specific promoter have been characterized and used to direct seed specific expression in transgenic tobacco plants (e.g. Vodkin, et al., Cell, 34: 1023, 1983; Lindstrom, et al., Developmental Genetics, 11: 160, 1990).

Particularly preferred promoters in include the light-inducible promoter from the small subunit of ribulose-1,5-bisphosphate carboxylase (ssRUB1SCO); the EIF-4A promoter from tobacco (Mandel, et al., Plant Mol. Biol, 29: 995-1004, 1995); the chitinase promoter from Arabidopsis (Samac, et al., Plant Cell, 3:1063-1072, 1991); the LTP (Lipid Transfer Protein) promoters from broccoli (Pyee, et al., Plant J., 7: 49-59, 1995); the petunia chalcone isomerase promoter (Van Tunen, et al., EMBO J. 7: 1257, 1988); the bean glycine rich protein 1 promoter (Keller, et al., EMBO L., 8: 1309-1314, 1989); and the potato patatin promoter (Wenzler, et al., Plant Mol. Biol., 12: 41-50, 1989).

Additional promoters include tobacco RB7, tobacco EIF-4, and lectin protein (Le1).

In a particular embodiment, the dicot promoter is selected from the group consisting of AtRd29A, Gm571, AtGolS3 and AtYP0104.

Any monocot promoter or virus promoter can provide the source of the sequence corresponding to the TATA box to transcription start site region used in the present invention. The monocot promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described (Poszkowski, et al., EMBO J., 3: 2719, 1989; Odell, et al., Nature, 313:810, 1985; Chau et al., Science, 244:174-181. 1989).

Often-used constitutive virus promoters include the CaMV 35S promoter (Odell, et al., Nature, 313: 810, 1985), the enhanced CaMV 35S promoter and the Figwort Mosaic Virus (FMV) promoter (Richins, et al., Nucleic Acids Res. 20: 8451, 1987.

Useful inducible promoters include promoters induced by salicylic acid or polyacrylic acids (PR-1; Williams, et al., Biotechnology 10:540-543, 1992), induced by application of safeners (substituted benzenesulfonamide herbicides; Hershey and Stoner, Plant Mol. Biol. 17: 679-690, 1991), heat-shock promoters (Ou-Lee et al., Proc. Natl. Acad. Sci. U.S.A. 83: 6815, 1986; Ainley et al., Plant Mol. Biol. 14: 949, 1990), a nitrate-inducible promoter derived from the spinach nitrite reductase transcribable polynucleotide sequence (Back et al., Plant Mol. Biol. 17: 9, 1991), hormone-inducible promoters (Yamaguchi-Shinozaki et al., Plant Mol. Biol. 15: 905, 1990), and light-inducible promoters associated with the small subunit of RuBP carboxylase and LHCP families (Kuhlemeier et al., Plant Cell 1: 471, 1989; Feinbaum et al., Mol. Gen. Genet. 226: 449-456, 1991; Weisshaar, et al., EMBO J. 10: 1777-1786, 1991; Lam and Chua, J. Biol. Chem. 266: 17131-17135, 1990; Castresana et al., EMBO J. 7: 1929-1936, 1988; Schulze-Lefert, et al., EMBO J. 8: 651, 1989).

Plant functional promoters useful for preferential expression in seed plastid include those from plant storage proteins and from proteins involved in fatty acid biosynthesis in oilseeds. Examples of such promoters include the 5′ regulatory regions from such transcribable polynucleotide sequences as napin (Kridl et al., Seed Sci. Res. 1: 209, 1991), phaseolin, zein, trypsin inhibitor, ACP, stearoyl-ACP desaturase, and oleosin. Seed-specific regulation is discussed in EP 0 255 378.

Another exemplary tissue-specific promoter is the lectin promoter, which is specific for seed tissue.

Particularly preferred promoters include the corn sucrose synthetase 1 (Yang, et al., Proc. Natl. Acad. Sci. USA, 87: 4144-48, 1990); corn alcohol dehydrogenase 1 (Vogel, et al., J. Cell Biochem., (Suppl) 13D: 312, 1989); corn light harvesting complex (Simpson, Science, 233: 34, 1986); corn heat shock protein (Odell, et al., Nature, 313: 810, 1985); the ubiquitin promoter from maize (Christensen et al., Plant Mol. Biol., 18: 675,689, 1992); and the actin promoter from corn (McElroy, et al., Plant Cell, 2:163-171, 1990).

Other promoters within the scope of this invention include seed selective, tissue specific, constitutive, or inducible promoters. For instance, the promoter may be the cauliflower mosaic virus 19S or 35S (CaMV19S, CaMV35S), enhanced CaMV (eCaMV), ribulose 1,5-bisphosphate carboxylase (ssRUBISCO), figwort mosaic virus (FMV), CaMV derived AS4, wheat PDX1, or corn RC2 promoter.

In particular embodiments, the promoter is the 35S promoter from CaMV or the Rice Actin1 promoter.

In another embodiment, the regulatory polynucleotide has a nucleic acid sequence that hybridizes to SEQ ID NO: 11 through SEQ ID NO: 17, or any complements thereof, or any cis elements thereof, or any fragments thereof.

The polynucleotide molecules of the present invention comprise chimeric gene expression elements engineered from monocot and dicot sources. Specifically, the chimeric molecules are engineered from dicot promoters that further comprise monocot region from the TATA box to the monocot Transcription Start Site (TSS), in place of the native dicot region from the TATA box to the dicot TSS. Examples of promoters of the present invention include those disclosed in Table 1.

The Transcription Start Site, or TSS, is defined as the point in a DNA sequence at which transcription of a gene into RNA begins. A promoter comprises specific DNA sequences that are recognized by proteins known as transcription factors, which bind to the promoter, recruiting RNA polymerase, the enzyme that synthesizes the RNA from the coding region of the gene. RNA polymerase binds to the DNA sequence at the promoter TSS.

TSS regions are generally known in the art. In the instance where a particular TSS region is unclear, the region can be experimentally validated by those skilled in the art by laboratory methods like primer extension and nuclear run off assays. For most plant promoters, the TSS is within a distance of 25-35 nucleotides from TATA box. Additionally, the TSS position can be predicted by computational methods.

As contemplated in this invention, nucleotide sequence 3′ to the TSS are included within the scope of this invention and the definition of “transcription start site.” In one embodiment, any sequence 3′ to the TSS up to the translation start can be included in the present invention. In another embodiment, sequences 3′ to the transcription start site called the “5′ UTR region” might also be included. In yet another embodiment, no sequence 3′ of the TSS is included.

TABLE 1
Sequence Annotations

| SEQ | | | Construct |
|---|---|---|---|
| ID | Sequence Name | Descriptions/Components | (pMON) ID |
| 1 | P-At.rd29A | native Arabidopsis rd29A promoter | 79353 |
| 2 | P-At.rd29A-TATA | native Arabidopsis rd29A promoter | |
| | | minus the TATA element and TSS | |
| 3 | P-At.GolS3 | native Arabidopsis GolS3 promoter | 79358 |
| 4 | P-At.GolS3-TATA | native Arabidopsis GolS3 promoter | |
| | | minus the TATA element and TSS | |
| 5 | P-At.YP0104 | native Arabidopsis YP0104 promoter | 79359 |
| 6 | P-At.YP0104-TATA | native Arabidopsis YP0104 promoter | |
| | | minus the TATA element and TSS | |
| 7 | P-Gm.700981571 | native Glycine max Gm571 promoter | 79361 |
| | (a.k.a. P-Gm.571) | | |
| 8 | P-Gm.700981571-TATA | native Glycine max Gm571 promoter | |
| | | minus the TATA element and TSS | |
| 9 | CaMV35S TATA | TATA element and TSS from the | |
| | | CaMV35S promoter | |
| 10 | Ract1 TATA | TATA element and TSS from the | |
| | | Rice Actin 1 promoter | |
| 11 | Chimeric | rd29A promoter minus native TATA + | 79354 |
| | At.rd29A/CaMV35S | CaMV35S promoter TATA element | |
| | promoter | | |
| 12 | Chimeric | rd29A promoter minus native TATA + | 79367 |
| | At.rd29A/Rice Actin 1 | Rice Actin 1 promoter TATA | |
| | promoter | element | |
| 13 | Chimeric | GolS3 promoter minus native TATA + | 79362 |
| | At.Go1S3/CaMV35S | CaMV35S promoter TATA element | |
| | promoter | | |
| 14 | Chimeric | YP0104 promoter minus native | 79356 |
| | At.YP0104/CaMV35S | TATA + CaMV35S promoter TATA | |
| | promoter | element | |
| 15 | Chimeric | YP0104 promoter minus native | 79365 |
| | At. YP0104/Rice Actin 1 | TATA + Rice Actin 1 promoter | |
| | promoter | TATA element | |
| 16 | Chimeric | Gm571 promoter minus native TATA + | 79360 |
| | Gm.571/CaMV35S | CaMV35S promoter TATA element | |
| | promoter | | |
| 17 | Chimeric | Gm571 promoter minus native TATA + | 79366 |
| | Gm.571/Rice Actin 1 | Rice Actin 1 promoter TATA | |
| | Promoter | element | |

As used in this invention, the “TATA box” includes sequences 5′ to the TATA box. While it is within the scope of this invention not to include any sequence 5′ to the putative TATA box, any small region, i.e. stretch of sequence, can be included. In one embodiment, 100 nucleotides 5′ of the putative TATA box are considered part of the TATA box, as used in this application. In another embodiment, 50 nucleotides 5′ of the putative TATA box are considered part of the TATA box. The TATA box is generally well known in the art and can be identified by those skilled in the art. The putative promoter sequences immediately upstream of the coding start site of the predicted genes within a given sequence size range, generally determines the location of the TATA box. Additionally, the transcription start site and TATA-box may be predicted with a program such as TSSP (SoftBerry, Inc., Mount Kisco, N.Y.). TSSP is designed for predicting PolII promoter regions in plants, and is based on a discriminate analysis combining characteristics of functional elements of regulatory sequences with the regulatory motifs from Softberry Inc.'s plant RegSite database (Solovyev V. V. (2001) Statistical approaches in Eukaryotic gene prediction. In: Handbook of Statistical genetics (eds. Balding D. et al.), John Wiley & Sons, Ltd., p. 83-127). In the cases that multiple TATA-boxes are predicted, only the rightmost (i.e. closest to the 5′ end) TATA-box is kept. The transcription start sites (TSS) are refined and extended upstream, based on the matches to the database sequences. Promoter sequences with unique TATA-box, as well the TATA-box locations, may be identified within the promoter sequences.

### Determination of Sequence Similarity Using Hybridization Techniques

Nucleic acid hybridization is a technique well known to those of skill in the art of DNA manipulation. The hybridization properties of a given pair of nucleic acids are an indication of their similarity or identity.

The term “hybridization” refers generally to the ability of nucleic acid molecules to join via complementary base strand pairing. Such hybridization may occur when nucleic acid molecules are contacted under appropriate conditions. “Specifically hybridizes” refers to the ability of two nucleic acid molecules to form an anti-parallel, double-stranded nucleic acid structure. A nucleic acid molecule is said to be the “complement” of another nucleic acid molecule if they exhibit “complete complementarity,” i.e., each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions are said to be “hybridizable cognates” of the other nucleic acid molecules. Conventional low stringency and high stringency conditions are described herein and by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are permissible, as long as such departures do not completely preclude the capacity of the molecules to form a double-stranded structure.

Low stringency conditions may be used to select nucleic acid sequences with lower sequence identities to a target nucleic acid sequence. One may wish to employ conditions such as about 0.15 M to about 0.9 M sodium chloride, at temperatures ranging from about 20° C. to about 55° C., for instance about 42° C. High stringency conditions may be used to select for nucleic acid sequences with higher degrees of identity to the disclosed nucleic acid sequences (Sambrook et al., 1989). High stringency conditions typically involve nucleic acid hybridization in about 2× to about 10×SSC (diluted from a 20×SSC stock solution containing 3 M sodium chloride and 0.3 M sodium citrate, pH 7.0 in distilled water), about 2.5× to about 5×Denhardt's solution (diluted from a 50× stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 50° C. to about 70° C., for instance about 55° C. or about 65° C., for several hours to overnight. High stringency conditions are preferably provided by 6×SSC, 5×Denhardt's solution, 100 mg/mL fish sperm DNA, and 0.1% (w/v) SDS, with an incubation at 55° C. for several hours. Hybridization is generally followed by several wash steps. The wash compositions generally comprise 0.5× to about 10×SSC, and 0.01% (w/v) to about 0.5% (w/v) SDS with a 15 minute incubation at about 20° C. to about 70° C. Preferably, the nucleic acid segments remain hybridized after washing at least one time in 0.1×SSC at 65° C.

A nucleic acid molecule preferably comprises a nucleic acid sequence that hybridizes, under low or high stringency conditions, with SEQ ID NO: 11 through SEQ ID NO: 17, any complements thereof, or any fragments thereof, or any cis elements thereof. A nucleic acid molecule most preferably comprises a nucleic acid sequence that hybridizes under high stringency conditions with SEQ ID NO: 11 through SEQ ID NO: 17, any complements thereof, or any fragments thereof, or any cis elements thereof.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity may be determined, for instance, using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, Journal of Molecular Biology 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). The percent identity is most preferably determined using the “Best Fit” program.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, NCBI, NLM, NIH; Altschul et al., J. Mol. Biol. 215:403-410 (1990). Version 2.0 or higher of BLAST allows the introduction of gaps (deletions and insertions) into alignments. For peptide sequence, BLASTX can be used to determine sequence identity. For polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 92%, 95%, 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 92%, 95%, 98% or about 99% sequence identity with a polynucleotide sequence described herein. Polynucleotide molecules that are capable of regulating transcription of operably linked transcribable polynucleotide molecules and have a substantial percent sequence identity to the polynucleotide sequences of the polynucleotide molecules provided herein are encompassed within the scope of this invention.

“Homology” is sometimes used to refer to the level of similarity between two or more nucleic acid or amino acid sequences in terms of percent of positional identity (i.e., sequence similarity or identity). Homology also refers to the concept of evolutionary relatedness, often evidenced by similar functional properties among different nucleic acids or proteins that share similar sequences.

In an alternative embodiment, the nucleic acid molecule comprises a nucleic acid sequence that exhibits 70% or greater identity, and more preferably at least 80% or greater, 85% or greater, 87% or greater, 88% or greater, 89% or greater, 90% or greater, 91% or greater, 92% or greater, 93% or greater, 94% or greater, 95% or greater, 96% or greater, 97% or greater, 98% or greater, or 99% or greater identity to a nucleic acid molecule selected from the group consisting of SEQ ID NO: 11 through SEQ ID NO: 17, any complement thereof, any fragment thereof, or any cis element thereof. The nucleic acid molecule preferably comprises a nucleic acid sequence that exhibits a 75% or greater sequence identity with a polynucleotide selected from the group consisting of SEQ ID NO: 11 through SEQ ID NO: 17, any complements thereof, any fragments thereof, or any cis elements thereof. The nucleic acid molecule more preferably comprises a nucleic acid sequence that exhibits an 80% or greater sequence identity with a polynucleotide selected from the group consisting of SEQ ID NO: 11 through SEQ ID NO: 17, any complements thereof, any fragments thereof, or any cis elements thereof. The nucleic acid molecule most preferably comprises a nucleic acid sequence that exhibits an 85% or greater sequence identity with a polynucleotide selected from the group consisting of SEQ ID NO: 11 through SEQ ID NO: 17, any complements thereof, any fragments thereof, or any cis elements thereof.

For purposes of this invention “percent identity” may also be determined using BLASTX version 2.0 for translated nucleotide sequences and BLASTN version 2.0 for polynucleotide sequences. In a preferred embodiment of the present invention, the presently disclosed corn genomic promoter sequences comprise nucleic acid molecules or fragments having a BLAST score of more than 200, preferably a BLAST score of more than 300, and even more preferably a BLAST score of more than 400 with their respective homologues.

### Polynucleotide Molecules, Motifs, Fragments, Chimeric Molecules

Nucleic acid molecules of the present invention include nucleic acid sequences that are between about 0.01 Kilobases (kb) and about 50 kb more preferably between about 0.1 kb and about 25 kb, even more preferably between about 1 kb and about 10 kb, and most preferably between about 3 kb and about 10 kb, about 3 kb and about 7 kb, about 4 kb and about 6 kb, about 2 kb and about 4 kb, about 2 kb and about 5 kb, about 1 kb and about 5 kb, about 1 kb and about 3 kb, or about 1 kb and about 2 kb.

As used herein, the term “fragment” or “fragment thereof” refers to a finite polynucleotide sequence length that comprises at least 25, at least 50, at least 75, at least 85, at least 95, at least 110, at least 125, at least 150, at least 175, at least 200, at least 250, at least 300 and at least 500 or more contiguous bases up to the full length of a referenced sequence provided herein.

### Regulatory Elements

Gene expression is finely regulated at both the transcriptional and post-transcriptional levels. A spectrum of control regions regulates transcription by RNA polymerase II. Enhancers that can stimulate transcription from a promoter tens of thousands of base pairs away (e.g., the SV40 enhancer) are an example of long-range effectors, whereas more proximal elements include promoters and introns. Transcription initiates at the cap site encoding the first nucleotide of the first exon of an mRNA. For many genes, especially those encoding abundantly expressed proteins, a TATA box located 25-30 base pairs upstream from the cap site directs RNA polymerase II to the start site. Promoter-proximal elements roughly within the first 200 base pairs upstream of the cap site stimulate transcription.

Features of the untranslated regions of mRNAs that control translation, degradation and localization include stem-loop structures, upstream initiation codons and open reading frames, internal ribosome entry sites and various cis-acting elements that are bound by RNA-binding proteins.

The present invention regulatory element sequences may comprise cis-elements, enhancers, terminators, or introns. Regulatory elements may be isolated or identified from untranslated regions (UTRs) from a particular polynucleotide sequence. Any of the regulatory elements described herein may be present in a recombinant construct of the present invention.

One skilled in the art would know various introns, enhancers, transit peptides, targeting signal sequences, 5′ and 3′ untranslated regions (UTRs), as well as other molecules involved in the regulation of gene expression that are useful in the design of effective plant expression vectors, such as those disclosed, for example, in U.S. Patent Application Publication 2003/01403641 (herein incorporated by reference).

### UTRs

UTRs are known to play crucial roles in the post-transcriptional regulation of gene expression, including modulation of the transport of mRNAs out of the nucleus and of translation efficiency, subcellular localization and stability. Regulation by UTRs is mediated in several ways. Nucleotide patterns or motifs located in 5′ UTRs and 3′ UTRs can interact with specific RNA-binding proteins. Unlike DNA-mediated regulatory signals, however, whose activity is essentially mediated by their primary structure, the biological activity of regulatory motifs at the RNA level relies on a combination of primary and secondary structure. Interactions between sequence elements located in the UTRs and specific complementary RNAs have also been shown to play key regulatory roles. Finally, there are examples of repetitive elements that are important for regulation at the RNA level, affecting translation efficiency.

For example, non-translated 5′ leader polynucleotide molecules derived from heat shock protein genes have been demonstrated to enhance gene expression in plants (see for example, U.S. Pat. No. 5,659,122 and U.S. Pat. No. 5,362,865, all of which are incorporated herein by reference).

### Cis-Acting Elements

Many regulatory elements act in cis (“cis elements”) and are believed to affect DNA topology, producing local conformations that selectively allow or restrict access of RNA polymerase to the DNA template or that facilitate selective opening of the double helix at the site of transcriptional initiation. C is elements occur within the 5′ UTR associated with a particular coding sequence, and are often found within promoters and promoter modulating sequences (inducible elements). C is elements can be identified using known cis elements as a target sequence or target motif in the BLAST programs of the present invention. Examples of cis-acting elements in the 5′ UTR associated with a polynucleotide coding sequence include, but are not limited to, promoters and enhancers.

### Promoters

Among the gene expression regulatory elements, the promoter plays a central role. Along the promoter, the transcription machinery is assembled and transcription is initiated. This early step is often rate-limiting relative to subsequent stages of protein production. Transcription initiation at the promoter may be regulated in several ways. For example, a promoter may be induced by the presence of a particular compound or external stimuli, express a gene only in a specific tissue, express a gene during a specific stage of development, or constitutively express a gene. Thus, transcription of a transgene may be regulated by operably linking the coding sequence to promoters with different regulatory characteristics. Accordingly, regulatory elements such as promoters, play a pivotal role in enhancing the agronomic, pharmaceutical or nutritional value of crops.

As used herein, the term “promoter” refers to a polynucleotide molecule comprising a nucleotide sequence that is involved in recognition and binding of RNA polymerase II and other proteins such as transcription factors (trans-acting protein factors that regulate transcription) to initiate transcription of an operably linked gene. A promoter may be isolated from the 5′ untranslated region (5′ UTR) of a genomic copy of a gene. Alternately, promoters may be synthetically produced or manipulated DNA elements. Promoters may be defined by their temporal, spatial, or developmental expression pattern. A promoter can be used as a regulatory element for modulating expression of an operably linked transcribable polynucleotide molecule. Promoters may themselves contain sub-elements such as cis-elements or enhancer domains that effect the transcription of operably linked genes. A “plant promoter” is a native or non-native promoter that is functional in plant cells. A plant promoter can be used as a 5′ regulatory element for modulating expression of an operably linked gene or genes. Plant promoters may be defined by their temporal, spatial, or developmental expression pattern.

Any of the nucleic acid molecules described herein may comprise nucleic acid sequences comprising promoters. Promoters of the present invention can include between about 300 bp upstream and about 10 kb upstream of the trinucleotide ATG sequence at the start site of a protein coding region. Promoters of the present invention can include between about 300 bp upstream and about 5 kb upstream of the trinucleotide ATG sequence at the start site of a protein coding region. In other embodiments, promoters of the present invention can include between about 300 bp upstream and about 2 kb upstream of the trinucleotide ATG sequence at the start site of a protein coding region. Promoters of the present invention typically include between about 300 bp upstream and about 1 kb upstream of the trinucleotide ATG sequence at the start site of a protein coding region. In many circumstances even less than a 300 bp promoter may be sufficient for some level of expression, although additional sequences may act to further regulate expression, for example, in response to biochemical, developmental or environmental signals.

The promoter of the present invention preferably transcribes a heterologous transcribable polynucleotide sequence at a high level in a plant. More preferably, the promoter hybridizes to a nucleic acid sequence selected from the group consisting of SEQ ID NO: 11 through SEQ ID NO: 17, or any complements thereof; or any fragments thereof. Suitable hybridization conditions include those described above. A nucleic acid sequence of the promoter of the present invention preferably hybridizes, under low or high stringency conditions, with SEQ ID NO: 11 through SEQ ID NO: 17, or any complements thereof. The promoter most preferably hybridizes under high stringency conditions to a nucleic acid sequence selected from the group consisting of SEQ ID NO: 11 through SEQ ID NO: 17, or any complements thereof.

In an alternative embodiment, the promoter comprises a nucleic acid sequence that exhibits 85% or greater identity, and more preferably at least 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, or 99% or greater identity to a nucleic acid sequence selected from the group consisting of SEQ ID NO: 11 through SEQ ID NO: 17, or complements or fragments thereof. The promoter most preferably comprises a nucleic acid sequence selected from the group consisting of SEQ ID NO: 11 through SEQ ID NO: 17, any complements thereof, or any fragments thereof.

As used herein, by the term “promoter” is also meant promoter fragments that have activity in regulating gene expression. Promoter fragments may also comprise regulatory elements such as enhancer domains, and may further be useful for constructing chimeric molecules. Fragments of SEQ ID NO: 1, as well as any other SEQ ID NO provided herein, may comprise, for instance, at least about 50, 95, 150, 250, 400, or 450 contiguous nucleotides of the referenced polynucleotide sequence, such as SEQ ID NO:1. In one embodiment, such a sequence comprises up to the full 504 nucleotides of SEQ ID NO: 1. Fragments of SEQ ID NO: 3 may comprise, for instance, at least about 50, 95, 150, 250, 400, 750 or 900 contiguous nucleotides of the polynucleotide sequence of SEQ ID NO: 3, up to the full 1069 nucleotides of SEQ ID NO: 3. Fragments of SEQ ID NO: 5 may comprise, for instance, at least about 50, 95, 150, 250, 400, 750 or 900 contiguous nucleotides of the polynucleotide sequence of SEQ ID NO: 5, up to the full 1003 nucleotides of SEQ ID NO: 5. Fragments of SEQ ID NO: 7 may comprise, for instance, at least about 50, 95, 150, 250, 400, 750 or 900 contiguous nucleotides of the polynucleotide sequence of SEQ ID NO: 7, up to the full 1195 nucleotides of SEQ ID NO: 7.

At least two types of information are useful in predicting promoter regions within a genomic DNA sequence. First, promoters may be identified on the basis of their sequence “content,” such as transcription factor binding sites and various known promoter motifs. (e.g. Stormo, Genome Research 10: 394-397 (2000)). Such signals may be identified by computer programs that identify sites associated with promoters, such as TATA boxes and transcription factor (TF) binding sites. Second, promoters may be identified on the basis of their “location,” i.e. their proximity to a known or suspected coding sequence. (Stormo, Genome Research 10: 394-397 (2000)). Promoters are typically found within a region of DNA extending approximately 150-1500 basepairs (bp) in the 5′ direction from the start codon of a coding sequence. Thus, promoter regions may be identified by locating the start codon of a coding sequence, and moving beyond the start codon in the 5′ direction to locate the promoter region.

Promoter sequence may be analyzed for the presence of common promoter sequence characteristics, such as a TATA-box and other known transcription factor binding site motifs. These motifs are not always found in every known promoter, nor are they necessary for promoter function, but when present, do indicate that a segment of DNA is a promoter sequence.

For identification of other known transcription factor binding motifs (such as a GC-box, CAAT-box, etc.), the promoter sequences immediately upstream of the coding start site of the predicted genes within a given sequence size range, as described above, are used. The known transcription factor binding motifs (except TATA-box) on the promoter sequences may be predicted with a program such as PromoterScan (Prestridge, J. Mol. Biol. 249: 923-32 (1995)). The identification of such motifs provide important information about the candidate promoter. For example, some motifs are associated with informative annotations such as (but not limited to) “light inducible binding site” or “stress inducible binding motif” and can be used to select with confidence a promoter that is able to confer light inducibility or stress inducibility to an operably-linked transgene, respectively.

Putative promoter sequences are also searched with matcorns for the GC box (factor name: V_GC_{—}01) and CCAAT box (factor name: F_HAP234_{—}01). The matcorns for the GC box and the CCAAT box are from Transfac. The algorithm that is used to annotate promoters searches for matches to both sequence motifs and matrix motifs. First, individual matches are found. For sequence motifs, a maximum number of mismatches are allowed. If the code M, R, W, S, Y, or K are listed in the sequence motif (each of which is a degenerate code for 2 nucleotides) ½ mismatch is allowed. If the code B, D, H, or V is listed in the sequence motif (each of which is a degenerate code for 3 nucleotides) ⅓ mismatch is allowed. Appropriate p values may be determined by simulation by generation of a 5 Mb length of random DNA with the same dinucleotide frequency as the test set, and from this test set the probability of a given matrix score was determined (number of hits/5e7). Once the individual hits are found, the putative promoter sequence is searched for clusters of hits in a 250 bp window. The score for a cluster is found by summing the negative natural log of the p value for each individual hit. Using simulations with 100 Mb lengths, the probability of a window having a cluster score greater than or equal to the given value is determined. Clusters with a p value more significant than p<1e-6 are reported. Effects of repetitive elements are screened. For matrix motifs, a p value cutoff is used on a matrix score. The matrix score is determined by adding the path of a given DNA sequence through a matrix. Appropriate p values are determined by simulation: 5 Mb lengths of random DNA with the same dinucleotide frequency as a test set are generated to test individual matrix hits, and 100 Mb lengths are used to test clusters. The probability of a given matrix score and the probability scores for clusters are determined, as are the sequence motifs. The usual cutoff for matcorns is 2.5e-4. No clustering was done for the GC box or CAAT box.

Examples of promoters include: those described in U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter, P-Zm.L3), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter, P-Cl.Gcx), and U.S. Pat. No. 7,151,204 (maize chloroplast aldolase promoter), all of which are incorporated herein by reference in their entirety.

Promoters of the present invention include homologues of cis elements known to effect gene regulation that show homology with the promoter sequences of the present invention. These cis elements include, but are not limited to, oxygen responsive cis elements (Cowen et al., J. Biol. Chem. 268:26904-26910 (1993)), light regulatory elements (Bruce and Quaill, Plant Cell 2 (11):1081-1089 (1990); Bruce et al., EMBO J. 10:3015-3024 (1991); Rocholl et al., Plant Sci. 97:189-198 (1994); Block et al., Proc. Natl. Acad. Sci. USA 87:5387-5391 (1990); Giuliano et al., Proc. Natl. Acad. Sci. USA 85:7089-7093 (1988); Staiger et al., Proc. Natl. Acad. Sci. USA 86:6930-6934 (1989); Izawa et al., Plant Cell 6:1277-1287 (1994); Menkens et al., Trends in Biochemistry 20:506-510 (1995); Foster et al., FASEB J. 8:192-200 (1994); Plesse et al., Mol Gen Gene 254:258-266 (1997); Green et al., EMBO J. 6:2543-2549 (1987); Kuhlemeier et al., Ann. Rev Plant Physiol. 38:221-257 (1987); Villain et al., J. Biol. Chem. 271:32593-32598 (1996); Lam et al., Plant Cell 2:857-866 (1990); Gilmartin et al., Plant Cell 2:369-378 (1990); Datta et al., Plant Cell 1:1069-1077 (1989); Gilmartin et al., Plant Cell 2:369-378 (1990); Castresana et al., EMBO J. 7:1929-1936 (1988); Ueda et al., Plant Cell 1:217-227 (1989); Terzaghi et al., Annu. Rev. Plant Physiol. Plant Mol. Biol. 46:445-474 (1995); Green et al., EMBO J. 6:2543-2549 (1987); Villain et al., J. Biol. Chem. 271:32593-32598 (1996); Tjaden et al., Plant Cell 6:107-118 (1994); Tjaden et al., Plant Physiol. 108:1109-1117 (1995); Ngai et al., Plant J. 12:1021-1234 (1997); Ngai et al., Plant J. 12:1021-1034 (1997)), elements responsive to gibberellin, (Muller et al., J. Plant Physiol. 145:606-613 (1995); Croissant et al., Plant Science 116:27-35 (1996); Lohmer et al., EMBO J. 10:617-624 (1991); Rogers et al., Plant Cell 4:1443-1451 (1992); Lanahan et al., Plant Cell 4:203-211 (1992); Skriver et al., Proc. Natl. Acad. Sci. USA 88:7266-7270 (1991); Huang et al., Plant Mol. Biol. 14:655-668 (1990), Gubler et al., Plant Cell 7:1879-1891 (1995)), elements responsive to abscisic acid, (Busk et al., Plant Cell 9:2261-2270 (1997); Guiltinan et al., Science 250:267-270 (1990); Shen et al., Plant Cell 7:295-307 (1995); Shen et al., Plant Cell 8:1107-1119 (1996); Seo et al., Plant Mol. Biol. 27:1119-1131 (1995); Marcotte et al., Plant Cell 1:969-976 (1989); Shen et al., Plant Cell 7:295-307 (1995); Iwasaki et al., Mol Gen Genet. 247:391-398 (1995); Hattori et al., Genes Dev. 6:609-618 (1992); Thomas et al., Plant Cell 5:1401-1410 (1993)), elements similar to abscisic acid responsive elements, (Ellerstrom et al., Plant Mol. Biol. 32:1019-1027 (1996)), auxin responsive elements (Liu et al., Plant Cell 6:645-657 (1994); Liu et al., Plant Physiol. 115:397-407 (1997); Kosugi et al., Plant J. 7:877-886 (1995); Kosugi et al., Plant Cell 9:1607-1619 (1997); Ballas et al., J. Mol. Biol. 233:580-596 (1993)), a cis element responsive to methyl jasmonate treatment (Beaudoin and Rothstein, Plant Mol. Biol. 33:835-846 (1997)), a cis element responsive to abscisic acid and stress response (Straub et al., Plant Mol. Biol. 26:617-630 (1994)), ethylene responsive cis elements (Itzhaki et al., Proc. Natl. Acad. Sci. USA 91:8925-8929 (1994); Montgomery et al., Proc. Natl. Acad. Sci. USA 90:5939-5943 (1993); Sessa et al., Plant Mol. Biol. 28:145-153 (1995); Shinshi et al., Plant Mol. Biol. 27:923-932 (1995)), salicylic acid cis responsive elements, (Strange et al., Plant J. 11:1315-1324 (1997); Qin et al., Plant Cell 6:863-874 (1994)), a cis element that responds to water stress and abscisic acid (Lam et al., J. Biol. Chem. 266:17131-17135 (1991); Thomas et al., Plant Cell 5:1401-1410 (1993); Pla et al., Plant Mol Biol 21:259-266 (1993)), a cis element essential for M phase-specific expression (Ito et al., Plant Cell 10:331-341 (1998)), sucrose responsive elements (Huang et al., Plant Mol. Biol. 14:655-668 (1990); Hwang et al., Plant Mol. Biol. 36:331-341 (1998); Grierson et al., Plant J. 5:815-826 (1994)), heat shock response elements (Pelham et al., Trends Genet. 1:31-35 (1985)), elements responsive to auxin and/or salicylic acid and also reported for light regulation (Lam et al., Proc. Natl. Acad. Sci. USA 86:7890-7897 (1989); Benfey et al., Science 250:959-966 (1990)), elements responsive to ethylene and salicylic acid (Ohme-Takagi et al., Plant Mol. Biol. 15:941-946 (1990)), elements responsive to wounding and abiotic stress (Loake et al., Proc. Natl. Acad. Sci. USA 89:9230-9234 (1992); Mhiri et al., Plant Mol. Biol. 33:257-266 (1997)), antoxidant response elements (Rushmore et al., J. Biol. Chem. 266:11632-11639; Dalton et al., Nucleic Acids Res. 22:5016-5023 (1994)), Sph elements (Suzuki et al., Plant Cell 9:799-807 1997)), elicitor responsive elements, (Fukuda et al., Plant Mol. Biol. 34:81-87 (1997); Rushton et al., EMBO J. 15:5690-5700 (1996)), metal responsive elements (Stuart et al., Nature 317:828-831 (1985); Westin et al., EMBO J. 7:3763-3770 (1988); Thiele et al., Nucleic Acids Res. 20:1183-1191 (1992); Faisst et al., Nucleic Acids Res. 20:3-26 (1992)), low temperature responsive elements, (Baker et al., Plant Mol. Biol. 24:701-713 (1994); Jiang et al., Plant Mol. Biol. 30:679-684 (1996); Nordin et al., Plant Mol. Biol. 21:641-653 (1993); Zhou et al., J. Biol. Chem. 267:23515-23519 (1992)), drought responsive elements, (Yamaguchi et al., Plant Cell 6:251-264 (1994); Wang et al., Plant Mol. Biol. 28:605-617 (1995); Bray EA, Trends in Plant Science 2:48-54 (1997)), enhancer elements for glutenin, (Colot et al., EMBO J. 6:3559-3564 (1987); Thomas et al., Plant Cell 2:1171-1180 (1990); Kreis et al., Philos. Trans. R. Soc. Lond., B314:355-365 (1986)), light-independent regulatory elements, (Lagrange et al., Plant Cell 9:1469-1479 (1997); Villain et al., J. Biol. Chem. 271:32593-32598 (1996)), OCS enhancer elements, (Bouchez et al., EMBO J. 8:4197-4204 (1989); Foley et al., Plant J. 3:669-679 (1993)), ACGT elements, (Foster et al., FASEB J. 8:192-200 (1994); Izawa et al., Plant Cell 6:1277-1287 (1994); Izawa et al., J. Mol. Biol. 230:1131-1144 (1993)), negative cis elements in plastid related genes, (Zhou et al., J. Biol. Chem. 267:23515-23519 (1992); Lagrange et al., Mol. Cell Biol. 13:2614-2622 (1993); Lagrange et al., Plant Cell 9:1469-1479 (1997); Zhou et al., J. Biol. Chem. 267:23515-23519 (1992)), prolamin box elements, (Forde et al., Nucleic Acids Res. 13:7327-7339 (1985); Colot et al., EMBO J. 6:3559-3564 (1987); Thomas et al., Plant Cell 2:1171-1180 (1990); Thompson et al., Plant Mol. Biol. 15:755-764 (1990); Vicente et al., Proc. Natl. Acad. Sci. USA 94:7685-7690 (1997)), elements in enhancers from the IgM heavy chain gene (Gillies et al., Cell 33:717-728 (1983); and Whittier et al., Nucleic Acids Res. 15:2515-2535 (1987)).

The activity or strength of a promoter may be measured in terms of the amount of mRNA or protein accumulation it specifically produces, relative to the total amount of mRNA or protein. The promoter preferably expresses an operably linked nucleic acid sequence at a level greater than 0.01%; preferably in a range of about 0.5% to about 20% (w/w) of the total cellular RNA or protein.

Alternatively, the activity or strength of a promoter may be expressed relative to a well-characterized promoter (for which transcriptional activity was previously assessed). For example, a less-characterized promoter may be operably linked to a reporter sequence (e.g., GUS) and introduced into a specific cell type. A well-characterized promoter (e.g. the 35S promoter) is similarly prepared and introduced into the same cellular context. Transcriptional activity of the unknown promoter is determined by comparing the amount of reporter expression, relative to the well characterized promoter. In one embodiment, the activity of the present promoter is as strong as the 35S promoter when compared in the same cellular context. The cellular context may be, for instance, rice, Arabidopsis, sorghum, corn, barley, wheat, canola, soybean, or maize.

### Enhancers

Enhancers, which strongly activate transcription, frequently in a specific differentiated cell type, are usually 100-200 base pairs long. Although enhancers often lie within a few kilobases of the cap site, in some cases they lie much further upstream or downstream from the cap site or within an intron. Some genes are controlled by more than one enhancer region, as in the case of the Drosophila even-skipped gene.

As used herein, the term “enhancer domain” refers to a cis-acting transcriptional regulatory element (cis-element), which confers an aspect of the overall modulation of gene expression. An enhancer domain may function to bind transcription factors, trans-acting protein factors that regulate transcription. Some enhancer domains bind more than one transcription factor, and transcription factors may interact with different affinities with more than one enhancer domain. Enhancer domains can be identified by a number of techniques, including deletion analysis, i.e., deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of an enhancer domain can be further studied by mutagenesis (or substitution) of one or more nucleotides or by other conventional methods. Enhancer domains can be obtained by chemical synthesis or by isolation from regulatory elements that include such elements, and they can be synthesized with additional flanking nucleotides that contain useful restriction enzyme sites to facilitate subsequence manipulation.

Translational enhancers may also be incorporated as part of a recombinant vector. Thus the recombinant vector may preferably contain one or more 5′ non-translated leader sequences which serve to enhance expression of the nucleic acid sequence. Such enhancer sequences may be desirable to increase or alter the translational efficiency of the resultant mRNA. Examples of other regulatory element 5′ nucleic acid leader sequences include dSSU 5′, PetHSP70 5′, and GmHSP17.9 5′. A translational enhancer sequence derived from the untranslated leader sequence from the mRNA of the coat protein gene of alfalfa mosaic virus coat protein gene, placed between the promoter and the gene, to increase translational efficiency, is described in U.S. Pat. No. 6,037,527, herein incorporated by reference. Thus, the design, construction, and use of enhancer domains according to the methods disclosed herein for modulating the expression of operably linked transcribable polynucleotide molecules are encompassed by the present invention.

### Leaders

As used herein, the term “leader” refers to a polynucleotide molecule isolated from the untranslated 5′ region (5′ UTR) of a genomic copy of a gene and defined generally as a segment between the transcription start site (TSS) and the coding sequence start site. Alternately, leaders may be synthetically produced or manipulated DNA elements. A “plant leader” is a native or non-native leader that is functional in plant cells. A plant leader can be used as a 5′ regulatory element for modulating expression of an operably linked transcribable polynucleotide molecule.

For example, non-translated 5′ leader polynucleotide molecules derived from heat shock protein genes have been demonstrated to enhance gene expression in plants (see for example, U.S. Pat. No. 5,659,122 and U.S. Pat. No. 5,362,865, all of which are incorporated herein by reference).

### Introns

As used herein, the term “intron” refers to a polynucleotide molecule that may be isolated or identified from the intervening sequence of a genomic copy of a gene and may be defined generally as a region spliced out during mRNA processing prior to translation. Alternately, introns may be synthetically produced or manipulated DNA elements. Introns may themselves contain sub-elements such as cis-elements or enhancer domains that effect the transcription of operably linked genes. A “plant intron” is a native or non-native intron that is functional in plant cells. A plant intron may be used as a regulatory element for modulating expression of an operably linked gene or genes. A polynucleotide molecule sequence in a recombinant construct may comprise introns. The introns may be heterologous with respect to the transcribable polynucleotide molecule sequence.

The transcribable polynucleotide molecule sequence in the recombinant vector may comprise introns. The introns may be heterologous with respect to the transcribable polynucleotide molecule sequence. Examples of regulatory element introns include the corn actin intron and the corn HSP70 intron (U.S. Pat. No. 5,859,347, herein incorporated by reference in its entirety).

### Terminators

The 3′ untranslated regions (3′ UTRs) of mRNAs are generated by specific cleavage and polyadenylation. A 3′ polyadenylation region means a DNA molecule linked to and located downstream of a structural polynucleotide molecule and includes polynucleotides that provide a polyadenylation signal and other regulatory signals capable of affecting transcription, mRNA processing or gene expression. PolyA tails are thought to function in mRNA stability and in initiation of translation.

As used herein, the term “terminator” refers to a polynucleotide sequence that may be isolated or identified from the 3′ untranslated region (3′UTR) of a transcribable gene, which functions to signal to RNA polymerase the termination of transcription. The polynucleotide sequences of the present invention may comprise terminator sequences.

Polyadenylation is the non-templated addition of a 50 to 200 nt chain of polyadenylic acid (polyA). Cleavage must precede polyadenylation. The polyadenylation signal functions in plants to cause the addition of polyadenylate nucleotides to the 3′ end of the mRNA precursor. The polyadenylation sequence can be derived from the natural gene, from a variety of plant genes, or from Agrobacterium T-DNA genes. Transcription termination often occurs at sites considerably downstream of the sites that, after polyadenylation, are the 3′ ends of most eukaryotic mRNAs.

Examples of 3′ UTR regions are the nopaline synthase 3′ region (nos 3′; Fraley, et al., Proc. Natl. Acad. Sci. USA 80: 4803-4807, 1983), wheat hsp17 (T-Ta.Hsp17), and T-Ps.RbcS2:E9 (pea rubisco small subunit), those disclosed in WO0011200A2 (herein incorporated by reference) and other 3′ UTRs known in the art can be tested and used in combination with a DHDPS or AK coding region, herein referred to as T-3′UTR. Another example of terminator regions is given in U.S. Pat. No. 6,635,806, herein incorporated by reference.

### Regulatory Element Isolation and Modification

Any number of methods well known to those skilled in the art can be used to isolate a polynucleotide molecule, or fragment thereof, disclosed in the present invention. For example, PCR (polymerase chain reaction) technology can be used to amplify flanking regions from a genomic library of a plant using publicly available sequence information. A number of methods are known to those of skill in the art to amplify unknown polynucleotide molecules adjacent to a core region of known polynucleotide sequence. Methods include but are not limited to inverse PCR (IPCR), vectorette PCR, Y-shaped PCR, and genome walking approaches. Polynucleotide fragments can also be obtained by other techniques such as by directly synthesizing the fragment by chemical means, as is commonly practiced by using an automated oligonucleotide synthesizer. For the present invention, the polynucleotide molecules were isolated from genomic DNA by designing oligonucleotide primers based on available sequence information and using PCR techniques.

As used herein, the term “isolated polynucleotide molecule” refers to a polynucleotide molecule at least partially separated from other molecules normally associated with it in its native state. In one embodiment, the term “isolated” is also used herein in reference to a polynucleotide molecule that is at least partially separated from nucleic acids which normally flank the polynucleotide in its native state. Thus, polynucleotides fused to regulatory or coding sequences with which they are not normally associated, for example as the result of recombinant techniques, are considered isolated herein. Such molecules are considered isolated even when present, for example in the chromosome of a host cell, or in a nucleic acid solution. The term “isolated” as used herein is intended to encompass molecules not present in their native state.

Those of skill in the art are familiar with the standard resource materials that describe specific conditions and procedures for the construction, manipulation, and isolation of macromolecules (e.g., polynucleotide molecules, plasmids, etc.), as well as the generation of recombinant organisms and the screening and isolation of polynucleotide molecules.

Short nucleic acid sequences having the ability to specifically hybridize to complementary nucleic acid sequences may be produced and utilized in the present invention. These short nucleic acid molecules may be used as probes to identify the presence of a complementary nucleic acid sequence in a given sample. Thus, by constructing a nucleic acid probe which is complementary to a small portion of a particular nucleic acid sequence, the presence of that nucleic acid sequence may be detected and assessed. Use of these probes may greatly facilitate the identification of transgenic plants which contain the presently disclosed nucleic acid molecules. The probes may also be used to screen cDNA or genomic libraries for additional nucleic acid sequences related or sharing homology to the presently disclosed promoters and transcribable polynucleotide sequences. The short nucleic acid sequences may be used as probes and specifically as PCR probes. A PCR probe is a nucleic acid molecule capable of initiating a polymerase activity while in a double-stranded structure with another nucleic acid. Various methods for determining the structure of PCR probes and PCR techniques exist in the art. Computer generated searches using programs such as Primer3 (Rozen & Skaletsky, Methods Mol. Biol. 132:365-386, 2000), STSPipeline (www-genome.wi.mit.edu/cgi-bin/www.STS_Pipeline), or GeneUp (Pesole, et al., BioTechniques 25:112-123, 1998), for example, can be used to identify potential PCR primers.

Alternatively, the short nucleic acid sequences may be used as oligonucleotide primers to amplify or mutate a complementary nucleic acid sequence using PCR technology. These primers may also facilitate the amplification of related complementary nucleic acid sequences (e.g. related nucleic acid sequences from other species).

The primer or probe is generally complementary to a portion of a nucleic acid sequence that is to be identified, amplified, or mutated. The primer or probe should be of sufficient length to form a stable and sequence-specific duplex molecule with its complement. The primer or probe preferably is about 10 to about 200 nucleotides long, more preferably is about 10 to about 100 nucleotides long, even more preferably is about 10 to about 50 nucleotides long, and most preferably is about 14 to about 30 nucleotides long. The primer or probe may be prepared by direct chemical synthesis, by PCR (See, for example, U.S. Pat. Nos. 4,683,195, and 4,683,202, each of which is herein incorporated by reference), or by excising the nucleic acid specific fragment from a larger nucleic acid molecule.

### Transcribable Polynucleotide Molecules

A regulatory element of the present invention may be operably linked to a transcribable polynucleotide sequence that is heterologous with respect to the regulatory element. The term “heterologous” refers to the relationship between two or more nucleic acid or protein sequences that are derived from different sources. For example, a promoter is heterologous with respect to a transcribable polynucleotide sequence if such a combination is not normally found in nature. In addition, a particular sequence may be “heterologous” with respect to a cell or organism into which it is inserted (i.e. does not naturally occur in that particular cell or organism). The transcribable polynucleotide molecule may be modified to provide various desirable features. For example, a transcribable polynucleotide molecule may be modified to increase the content of essential amino acids, enhance translation of the amino acid sequence, alter post-translational modifications (e.g., phosphorylation sites), transport a translated product to a compartment inside or outside of the cell, improve protein stability, insert or delete cell signaling motifs, etc.

The transcribable polynucleotide molecule may generally be any nucleic acid sequence for which an increased level of transcription is desired. Alternatively, the regulatory element and transcribable polynucleotide sequence may be designed to down-regulate a specific nucleic acid sequence. This is typically accomplished by linking the promoter to a transcribable polynucleotide sequence that is oriented in the antisense direction. One of ordinary skill in the art is familiar with such antisense technology. Briefly, as the antisense nucleic acid sequence is transcribed, it hybridizes to and sequesters a complimentary nucleic acid sequence inside the cell. This duplex RNA molecule cannot be translated into a protein by the cell's translational machinery. Any nucleic acid sequence may be negatively regulated in this manner.

Due to the degeneracy of the genetic code, different nucleotide codons may be used to code for a particular amino acid. A host cell often displays a preferred pattern of codon usage. Transcribable polynucleotide molecules are preferably constructed to utilize the codon usage pattern of the particular host cell or to avoid rarely used sequence patterns. This generally enhances the expression of the transcribable polynucleotide sequence in a transformed host cell. Any of the above described nucleic acid and amino acid sequences may be modified to reflect the preferred codon usage of a host cell or organism in which they are contained. Modification of a transcribable polynucleotide sequence for optimal codon usage in plants is described in U.S. Pat. No. 5,689,052, herein incorporated by reference.

Additional variations in the transcribable polynucleotide molecules may encode proteins having equivalent or superior characteristics when compared to the proteins from which they are engineered. Mutations may include, but are not limited to, deletions, insertions, truncations, substitutions, fusions, shuffling of motif sequences, and the like. Mutations to a transcribable polynucleotide molecule may be introduced in either a specific or random manner, both of which are well known to those of skill in the art of molecular biology.

Thus, one embodiment of the invention is a regulatory element such as provided in SEQ ID NO: 11 through SEQ ID NO: 17, operably linked to a transcribable polynucleotide molecule so as to modulate transcription of said transcribable polynucleotide molecule at a desired level or in a desired tissue or developmental pattern upon introduction of said construct into a plant cell. In one embodiment, the transcribable polynucleotide molecule comprises a protein-coding region of a gene, and the regulatory element affects the transcription of a functional mRNA molecule that is translated and expressed as a protein product. In another embodiment, the transcribable polynucleotide molecule comprises an antisense region of a gene, and the regulatory element affects the transcription of an antisense RNA molecule or other similar inhibitory RNA in order to inhibit expression of a specific RNA molecule of interest in a target host cell.

### Genes of Agronomic Interest

The transcribable polynucleotide molecule preferably encodes a polypeptide that is suitable for incorporation into the diet of a human or an animal. Specifically, such transcribable polynucleotide molecules comprise genes of agronomic interest. As used herein, the term “gene of agronomic interest” refers to a transcribable polynucleotide molecule that includes but is not limited to a gene that provides a desirable characteristic associated with plant morphology, physiology, growth and development, yield, nutritional enhancement, disease or pest resistance, or environmental or chemical tolerance. Suitable transcribable polynucleotide molecules include but are not limited to those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, or an insecticidal protein.

In one embodiment of the invention, a polynucleotide molecule as shown in SEQ ID NO: 11 through SEQ ID NO: 17, or complements thereof, or fragments thereof, or cis elements thereof comprising regulatory elements is incorporated into a construct such that a polynucleotide molecule of the present invention is operably linked to a transcribable polynucleotide molecule that is a gene of agronomic interest.

The expression of a gene of agronomic interest is desirable in order to confer an agronomically important trait. A gene of agronomic interest that provides a beneficial agronomic trait to crop plants may be, for example, including, but not limited to genetic elements comprising herbicide resistance (U.S. Pat. Nos. 6,803,501; 6,448,476; 6,248,876; 6,225,114; 6,107,549; 5,866,775; 5,804,425; 5,633,435; 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; 6,506,962), virus resistance (U.S. Pat. Nos. 6,617,496; 6,608,241; 6,015,940; 6,013,864; 5,850,023; 5,304,730), nematode resistance (U.S. Pat. No. 6,228,992), bacterial disease resistance (U.S. Pat. No. 5,516,671), plant growth and development (U.S. Pat. No. 6,723,897; 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; 6,380,462), high oil production (U.S. Pat. Nos. 6,495,739; 5,608,149; 6,483,008; 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; 6,171,640), biopolymers (U.S. Pat. No. RE37,543; 6,228,623; 5,958,745 and U.S. Patent Publication No. US20030028917), environmental stress resistance (U.S. Pat. No. 6,072,103), pharmaceutical peptides and secretable peptides (U.S. Pat. Nos. 6,812,379; 6,774,283; 6,140,075; 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; 5,869,720) and biofuel production (U.S. Pat. No. 5,998,700). The genetic elements, methods, and transgenes described in the patents listed above are incorporated herein by reference.

Alternatively, a transcribable polynucleotide molecule can effect the above mentioned plant characteristic or phenotype by encoding a RNA molecule that causes the targeted inhibition of expression of an endogenous gene, for example via antisense, inhibitory RNA (RNAi), or cosuppression-mediated mechanisms. The RNA could also be a catalytic RNA molecule (i.e., a ribozyme) engineered to cleave a desired endogenous mRNA product. Thus, any transcribable polynucleotide molecule that encodes a transcribed RNA molecule that affects a phenotype or morphology change of interest may be useful for the practice of the present invention.

### Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding β-glucuronidase (GUS described in U.S. Pat. No. 5,599,670, which is incorporated herein by reference), green fluorescent protein (GFP described in U.S. Pat. No. 5,491,084 and U.S. Pat. No. 6,146,826, all of which are incorporated herein by reference), proteins that confer antibiotic resistance, or proteins that confer herbicide tolerance. Marker genes in genetically modified plants are generally of two types: genes conferring antibiotic resistance or genes conferring herbicide tolerance.

Useful antibiotic resistance markers, including those encoding proteins conferring resistance to kanamycin (nptII), hygromycin B (aphIV), streptomycin or spectinomycin (aad, spec/strep) and gentamycin (aac3 and aacC4) are known in the art.

Herbicides for which transgenic plant tolerance has been demonstrated and the method of the present invention can be applied, include but are not limited to: glyphosate, glufosinate, sulfonylureas, imidazolinones, bromoxynil, dalapon, dicamba, cyclohexanedione, protoporphyrinogen oxidase inhibitors, and isoxaflutole herbicides. Polynucleotide molecules encoding proteins involved in herbicide tolerance are known in the art, and include, but are not limited to a polynucleotide molecule encoding 5-enolpyruvylshikimate-3-phosphate synthase (EPSPS described in U.S. Pat. No. 5,627,061, U.S. Pat. No. 5,633,435, U.S. Pat. No. 6,040,497 and in U.S. Pat. No. 5,094,945 for glyphosate tolerance, all of which are incorporated herein by reference); polynucleotides encoding a glyphosate oxidoreductase and a glyphosate-N-acetyl transferase (GOX described in U.S. Pat. No. 5,463,175 and GAT described in U.S. Patent publication 20030083480, dicamba monooxygenase U.S. Patent publication 20030135879, all of which are incorporated herein by reference); a polynucleotide molecule encoding bromoxynil nitrilase (Bxn described in U.S. Pat. No. 4,810,648 for Bromoxynil tolerance, which is incorporated herein by reference); a polynucleotide molecule encoding phytoene desaturase (crtI) described in Misawa et al, (1993) Plant J. 4:833-840 and Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for tolerance to sulfonylurea herbicides; and the bar gene described in DeBlock, et al. (1987) EMBO J. 6:2513-2519 for glufosinate and bialaphos tolerance. The regulatory elements of the present invention can express transcribable polynucleotide molecules that encode for phosphinothricin acetyltransferase, glyphosate resistant EPSPS, aminoglycoside phosphotransferase, hydroxyphenyl pyruvate dehydrogenase, hygromycin phosphotransferase, neomycin phosphotransferase, dalapon dehalogenase, bromoxynil resistant nitrilase, anthranilate synthase, glyphosate oxidoreductase and glyphosate-N-acetyl transferase.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g., by ELISA), small active enzymes which are detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable marker genes will be apparent to those of skill in the art.

The selectable marker is preferably GUS, green fluorescent protein (GFP), neomycin phosphotransferase II (nptII), luciferase (LUX), an antibiotic resistance coding sequence, or an herbicide (e.g., glyphosate) resistance coding sequence. The selectable marker is most preferably a kanamycin, hygromycin, or herbicide resistance marker.

### Constructs and Vectors

The constructs of the present invention are generally double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the Agrobacterium cells, permit the integration of the T-DNA into the genome of a plant cell (see for example U.S. Pat. No. 6,603,061, herein incorporated by reference in its entirety). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, or LBA4404, however, other strains known to those skilled in the art of plant transformation can function in the present invention.

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908, 4,940,835, 4,769,061 and 4,757,011, all of which are herein incorporated by reference in their entirety. These type of vectors have also been reviewed (Rodriguez, et al. Vectors: A Survey of Molecular Cloning Vectors and Their Uses, Butterworths, Boston, 1988; Glick et al., Methods in Plant Molecular Biology and Biotechnology, CRC Press, Boca Raton, Fla., 1993). Typical vectors useful for expression of nucleic acids in higher plants are well known in the art and include vectors derived from the tumor-inducing (Ti) plasmid of Agrobacterium tumefaciens (Rogers, et al., Meth. Enzymol., 153:253-277, 1987). Other recombinant vectors useful for plant transformation, including the pCaMVCN transfer control vector, have also been described (Fromm et al., Proc. Natl. Acad. Sci. USA, 82(17): 5824-5828, 1985).

Various untranslated regulatory sequences may be included in the recombinant vector. Any such regulatory sequences may be provided in a recombinant vector with other regulatory sequences. Such combinations can be designed or modified to produce desirable regulatory features. Constructs of the present invention would typically comprise one or more gene expression regulatory elements operably linked to a transcribable polynucleotide molecule operably linked to a 3′ transcription termination polynucleotide molecule.

Constructs of the present invention may also include additional 5′ untranslated regions (5′ UTR) of an mRNA polynucleotide molecule or gene which can play an important role in translation initiation. For example, non-translated 5′ leader polynucleotide molecules derived from heat shock protein genes have been demonstrated to enhance gene expression in plants (see for example, U.S. Pat. No. 5,659,122 and U.S. Pat. No. 5,362,865, all of which are incorporated herein by reference). These additional upstream regulatory polynucleotide molecules may be derived from a source that is native or heterologous with respect to the other elements present on the construct.

One or more additional promoters may also be provided in the recombinant vector. These promoters may be operably linked to any of the transcribable polynucleotide sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Promoters which function in bacteria, yeast, and plants are all well taught in the art. The additional promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described (Poszkowski, et al., EMBO J., 3: 2719, 1989; Odell, et al., Nature, 313:810, 1985; Chau et al., Science, 244:174-181. 1989).

The promoter in the recombinant vector is preferably operably linked to a transcribable polynucleotide sequence. Exemplary transcribable polynucleotide sequences, and modified forms thereof, are described in detail above. The promoter of the present invention may be operably linked to a transcribable polynucleotide sequence that is heterologous with respect to the promoter. In one aspect, the transcribable polynucleotide sequence may generally be any nucleic acid sequence for which an increased level of transcription is desired. The transcribable polynucleotide sequence preferably encodes a polypeptide that is suitable for incorporation into the diet of a human or an animal. Suitable transcribable polynucleotide sequences include those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, and an insecticidal protein.

Alternatively, the promoter and transcribable polynucleotide sequence may be designed to down-regulate a specific nucleic acid sequence. This is typically accomplished by linking the promoter to a transcribable polynucleotide sequence that is oriented in the antisense direction. One of ordinary skill in the art is familiar with such antisense technology. Using such an approach, a cellular nucleic acid sequence is effectively down regulated as the subsequent steps of translation are disrupted. Nucleic acid sequences may be negatively regulated in this manner.

Methods are known in the art for constructing and introducing constructs into a cell in such a manner that the transcribable polynucleotide molecule is transcribed into a molecule that is capable of causing gene suppression. For example, post-transcriptional gene suppression using a construct with an anti-sense oriented transcribable polynucleotide molecule to regulate gene expression in plant cells is disclosed in U.S. Pat. No. 5,107,065 and U.S. Pat. No. 5,759,829; post-transcriptional gene suppression using a construct with a sense-oriented transcribable polynucleotide molecule to regulate gene expression in plants is disclosed in U.S. Pat. No. 5,283,184 and U.S. Pat. No. 5,231,020, all of which are hereby incorporated by reference.

Thus, one embodiment of the invention is a construct comprising a regulatory element such as provided in SEQ ID NO: 11 through SEQ ID NO: 17, operably linked to a transcribable polynucleotide molecule so as to modulate transcription of said transcribable polynucleotide molecule at a desired level or in a desired tissue or developmental pattern upon introduction of said construct into a plant cell. In one embodiment, the transcribable polynucleotide molecule comprises a protein-coding region of a gene, and the regulatory element affects the transcription of a functional mRNA molecule that is translated and expressed as a protein product. In another embodiment, the transcribable polynucleotide molecule comprises an antisense region of a gene, and the regulatory element affects the transcription of an antisense RNA molecule or other similar inhibitory RNA in order to inhibit expression of a specific RNA molecule of interest in a target host cell.

Exemplary transcribable polynucleotide molecules for incorporation into constructs of the present invention include, for example, polynucleotide molecules or genes from a species other than the target species or genes that originate with or are present in the same species, but are incorporated into recipient cells by genetic engineering methods rather than classical reproduction or breeding techniques. The type of polynucleotide molecule can include but is not limited to a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

Constructs comprising a chimeric regulatory element of the present invention may further comprise one or more transcribable polynucleotide molecules. In one embodiment of the invention, a polynucleotide molecule as shown in SEQ ID NO: 11 through SEQ ID NO: 17, or any complements thereof, or any fragments thereof, comprising regulatory elements such as promoters, is incorporated into a construct such that a polynucleotide molecule of the present invention is operably linked to a transcribable polynucleotide molecule that is a selectable marker or a gene of agronomic interest.

The gene regulatory elements of the present invention can be incorporated into a construct using selectable markers and tested in transient or stable plant analyses to provide an indication of the regulatory element's gene expression pattern in stable transgenic plants. Current methods of generating transgenic plants employ a selectable marker gene which is transferred along with any other genes of interest usually on the same DNA molecule. The presence of a suitable marker is necessary to facilitate the detection of genetically modified plant tissue during development.

Thus, in one embodiment of the invention, a polynucleotide molecule of the present invention as shown in SEQ ID NO: 11 through SEQ ID NO: 17, or fragments thereof, or complements thereof, or cis elements thereof is incorporated into a polynucleotide construct such that a polynucleotide molecule of the present invention is operably linked to a transcribable polynucleotide molecule that provides for a selectable, screenable, or scorable marker. The constructs containing the regulatory elements operably linked to a marker gene may be delivered to the tissues and the tissues analyzed by the appropriate mechanism, depending on the marker. The quantitative or qualitative analyses are used as a tool to evaluate the potential expression profile of a regulatory element when operatively linked to a gene of agronomic interest in stable plants. Any marker gene, described above, may be used in a transient assay.

Methods of testing for marker gene expression in transient assays are known to those of skill in the art. Transient expression of marker genes has been reported using a variety of plants, tissues, and DNA delivery systems. For example, types of transient analyses can include but are not limited to direct gene delivery via electroporation or particle bombardment of tissues in any transient plant assay using any plant species of interest. Such transient systems would include but are not limited to electroporation of protoplasts from a variety of tissue sources or particle bombardment of specific tissues of interest. The present invention encompasses the use of any transient expression system to evaluate regulatory elements operably linked to any transcribable polynucleotide molecule, including but not limited to marker genes or genes of agronomic interest. Examples of plant tissues envisioned to test in transients via an appropriate delivery system would include but are not limited to leaf base tissues, callus, cotyledons, roots, endosperm, embryos, floral tissue, pollen, and epidermal tissue.

### Transformation

The invention is also directed to a method of producing transformed cells and plants which comprise, in a 5′ to 3′ orientation, a gene expression regulatory element operably linked to a heterologous transcribable polynucleotide sequence. Other sequences may also be introduced into the cell, including 3′ transcriptional terminators, 3′ polyadenylation signals, other translated or untranslated sequences, transit or targeting sequences, selectable markers, enhancers, and operators.

The term “transformation” refers to the introduction of nucleic acid into a recipient host. The term “host” refers to bacteria cells, fungi, protests, animals and animal cells, plants and plant cells, or any plant parts or tissues including protoplasts, calli, roots, tubers, seeds, stems, leaves, seedlings, embryos, and pollen. As used herein, the term “transformed” refers to a cell, tissue, organ, or organism into which has been introduced a foreign polynucleotide molecule, such as a construct. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic plant as a parent in a cross and exhibiting an altered phenotype resulting from the presence of a foreign polynucleotide molecule. The term “transgenic” refers to an animal, plant, or other organism containing one or more heterologous nucleic acid sequences. The transformed cell or organism may include a rice, sorghum, barley, wheat, turfgrass, switchgrass, maize, or other member of the Poaceae, or Arabidopsis, canola, or soybean cell or plant, among others.

There are many methods for introducing nucleic acids into plant cells. The method generally comprises the steps of selecting a suitable host cell, transforming the host cell with a recombinant vector, and obtaining the transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol., 42: 205, 1991).

Technology for introduction of DNA into cells is well known to those of skill in the art. Methods and materials for transforming plant cells by introducing a plant polynucleotide construct into a plant genome in the practice of this invention can include any of the well-known and demonstrated methods including:
(1) Chemical methods (Graham and Van der Eb, Virology, 54(2): 536-539, 1973; Zatloukal, et al., Ann. N.Y. Acad. Sci., 660: 136-153, 1992);

(2) Physical methods such as microinjection (Capecchi, Cell, 22(2): 479-488, 1980), electroporation (Wong and Neumann, Biochim. Biophys. Res. Commun., 107(2): 584-587, 1982; Fromm et al., Proc. Natl. Acad. Sci. USA, 82(17): 5824-5828, 1985; U.S. Pat. No. 5,384,253, herein incorporated by reference) particle acceleration (Johnston and Tang, Methods Cell Biol., 43(A): 353-365, 1994; Fynan et al., Proc. Natl. Acad. Sci. USA, 90(24): 11478-11482, 1993) and microprojectile bombardment (as illustrated in U.S. Pat. Nos. 5,015,580; U.S. Pat. No. 5,550,318; U.S. Pat. No. 5,538,880; U.S. Pat. No. 6,160,208; U.S. Pat. No. 6,399,861; and U.S. Pat. No. 6,403,865, all of which are herein incorporated by reference);

(3) viral vectors (Clapp, Clin. Perinatol., 20(1): 155-168, 1993; Lu, et al., J. Exp. Med., 178(6): 2089-2096, 1993; Eglitis and Anderson, Biotechniques, 6(7): 608-614, 1988);

(4) receptor-mediated mechanisms (Curiel et al., Hum. Gen. Ther., 3(2):147-154, 1992; Wagner, et al., Proc. Natl. Acad. Sci. USA, 89(13): 6099-6103, 1992), and

(5) bacterial mediated mechanisms such as Agrobacterium-mediated transformation (as illustrated in U.S. Pat. No. 5,824,877; U.S. Pat. No. 5,591,616; U.S. Pat. No. 5,981,840; and U.S. Pat. No. 6,384,301, all of which are herein incorporated by reference);

(6) Nucleic acids can be directly introduced into pollen by directly injecting a plant's reproductive organs (Zhou, et al., Methods in Enzymology, 101: 433, 1983; Hess, Intern Rev. Cytol., 107: 367, 1987; Luo, et al., Plant Mol. Biol. Reporter, 6: 165, 1988; Pena, et al., Nature, 325: 274, 1987).

(7) Protoplast transformation, as illustrated in U.S. Pat. No. 5,508,184 (herein incorporated by reference).

(8) The nucleic acids may also be injected into immature embryos (Neuhaus, et al., Theor. Appl. Genet., 75: 30, 1987).

Any of the above described methods may be utilized to transform a host cell with one or more gene regulatory elements of the present invention and one or more transcribable polynucleotide molecules. A preferred embodiment of the present invention is the transformation of a plant cell. A plant transformation construct comprising a regulatory element of the present invention may be introduced into plants by any plant transformation method.

Methods for transforming dicotyledons, primarily by use of Agrobacterium tumefaciens and obtaining transgenic plants have been published for cotton (U.S. Pat. No. 5,004,863; U.S. Pat. No. 5,159,135; U.S. Pat. No. 5,518,908, all of which are herein incorporated by reference); soybean (U.S. Pat. No. 5,569,834; U.S. Pat. No. 5,416,011, all of which are herein incorporated by reference; McCabe, et al., Biotechnology, 6: 923, 1988; Christou et al., Plant Physiol. 87:671-674 (1988)); Brassica (U.S. Pat. No. 5,463,174, herein incorporated by reference); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258 (1995)).

Transformation of monocotyledons using electroporation, particle bombardment and Agrobacterium have also been reported. Transformation and plant regeneration have been achieved in asparagus (Bytebier et al., Proc. Natl. Acad. Sci. (USA) 84:5354 (1987)); barley (Wan and Lemaux, Plant Physiol 104:37 (1994)); maize (Rhodes et al., Science 240:204 (1988); Gordon-Kamm et al., Plant Cell 2:603-618 (1990); Fromm et al., Bio/Technology 8:833 (1990); Koziel et al., Bio/Technology 11:194 (1993); Armstrong et al., Crop Science 35:550-557 (1995)); oat (Somers et al., Bio/Technology 10:1589 (1992)); orchard grass (Horn et al., Plant Cell Rep. 7:469 (1988)); corn (Toriyama et al., Theor Appl. Genet. 205:34 (1986); Part et al., Plant Mol. Biol. 32:1135-1148 (1996); Abedinia et al., Aust. J. Plant Physiol. 24:133-141 (1997); Zhang and Wu, Theor. Appl. Genet. 76:835 (1988); Zhang et al., Plant Cell Rep. 7:379 (1988); Battraw and Hall, Plant Sci. 86:191-202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)) and wheat (Vasil et al., Bio/Technology 10:667 (1992); U.S. Pat. No. 5,631,152, herein incorporated by reference).

The regeneration, development, and cultivation of plants from transformed plant protoplast or explants is well taught in the art (Weissbach and Weissbach, Methods for Plant Molecular Biology, (Eds.), Academic Press, Inc., San Diego, Calif., 1988; Horsch et al., Science, 227: 1229-1231, 1985). In this method, transformants are generally cultured in the presence of a selective media which selects for the successfully transformed cells and induces the regeneration of plant shoots (Fraley et al., Proc. Natl. Acad. Sci. U.S.A., 80: 4803, 1983). These shoots are typically obtained within two to four months.

The shoots are then transferred to an appropriate root-inducing medium containing the selective agent and an antibiotic to prevent bacterial growth. Many of the shoots will develop roots. These are then transplanted to soil or other media to allow the continued development of roots. The method, as outlined, will generally vary depending on the particular plant strain employed.

The regenerated transgenic plants are self-pollinated to provide homozygous transgenic plants. Alternatively, pollen obtained from the regenerated transgenic plants may be crossed with non-transgenic plants, preferably inbred lines of agronomically important species. Conversely, pollen from non-transgenic plants may be used to pollinate the regenerated transgenic plants.

The transformed plants are analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays.

The seeds of the plants of this invention can be harvested from fertile transgenic plants and be used to grow progeny generations of transformed plants of this invention including hybrid plant lines comprising the construct of this invention and expressing a gene of agronomic interest. The present invention also provides for parts of the plants of the present invention. Plant parts, without limitation, include seed, endosperm, ovule and pollen. In a particularly preferred embodiment of the present invention, the plant part is a seed. The invention also includes and provides transformed plant cells which comprise a nucleic acid molecule of the present invention.

The transgenic plant may pass along the transformed nucleic acid sequence to its progeny. The transgenic plant is preferably homozygous for the transformed nucleic acid sequence and transmits that sequence to all of its offspring upon as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immunoprecipitation, and ELISA.

Having now generally described the invention, the same will be more readily understood through reference to the following examples which are provided by way of illustration, and are not intended to be limiting of the present invention, unless specified.

Each periodical, patent, and other document or reference cited herein is herein incorporated by reference in its entirety.

The following examples are included to demonstrate preferred embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1

### Promoter Isolation and Cloning Strategies

This example describes the materials and strategies for gene cloning and preparing a chimeric promoter construct in which the native region from the TATA box to the transcription start site (TSS) is substituted with a region from the TATA box to the TSS of another regulatory promoter. It also describes the method of preparing constructs with native promoter.

The base vector pMON77955 has provision to drop a promoter in a multiple cloning site just in front of the coding region of a reporter gene, such as CR-Ec.uidA. This vector was then completed with NOS 3′UTR and a kanamycin selection cassette using methods well known in the art.

The CaMV35S TATA box to TSS fragment (SEQ ID NO: 9), that was used for making chimeric constructs, was isolated by digesting pMON51011 with Hind III and BglII. This 80 bp fragment was then end filled and dropped in the StuI site of base vector pMON77955 and the resulting construct used to generate chimeric promoters with 35S TATA box to TSS. The Os.Act1 TATA box to TSS fragment (SEQ ID NO: 10) that is used for making chimeric constructs, was obtained by annealing synthetic oligonucleotide primers 1 & 2 (SEQ ID NOs:26-27) for the forward and reverse strand. This double stranded 80 bp fragment was also dropped in the StuI site of base vector pMON77955 and the resulting construct used to generate promoters with Os.Act1 TATA box to TSS. For all the promoter constructs the TATA box to TSS-lacking versions were PCR amplified and were then sub-cloned into pMON99667 (for promoter with 35S TATA box to TSS version) or pMON99668 (for promoter with Os.Act1 TATA box to TSS).

The native Arabidopsis promoter rd29A (P-At.rd29A, SEQ ID NO: 1) was PCR amplified from genomic DNA using primer 3 and primer 4, utilizing methods well known in the art. P-At.rd29A full length was extracted from pMON57270 as a Not I-EcoR I fragment and was cloned into Not I-EcoR I sites of pMON77951 resulting into pMON79353 which was used for transformation.

P-At.rd29A without its native TATA box to TSS (SEQ ID NO: 2) was PCR amplified from Arabidopsis genomic DNA using primers 5 & 6 and was cloned into Kpn I site of pMON99667 to generate the construct pMON79354, comprising the engineered P-At.rd29A with the 35S TATA box to TSS (SEQ ID NO: 11) which was used for transformation.

P-At.rd29A without its native TATA box to TSS (SEQ ID NO: 2) was PCR amplified from Arabidopsis genomic DNA using primers 5 & 6 and was cloned into Kpn I site of pMON99668 to generate a promoter construct pMON79367, comprising the engineered P-At.rd29A with the Os.Act1 TATA box to TSS (SEQ ID NO: 12) which was used for transformation.

The native Arabidopsis promoter GolS3 (P-At.GolS3, SEQ ID NO: 3) was PCR amplified from Arabidopsis genomic DNA using primers 7 & 8 and was cloned in pMON77955 at BamH I-Stu I site resulting in a construct pMON79358 which was used for transformation.

P-At.GolS3 without its native TATA box to TSS (SEQ ID NO: 4) was PCR amplified from Arabidopsis genomic DNA using Primers 9 & 10 and was cloned into Xho I site of pMON99667 to generate a promoter construct pMON79362, comprising the engineered P-At.GolS3 with the 35S TATA box to TSS (SEQ ID NO: 13) which was used for transformation.

The native Arabidopsis promoter YP0104 (P-At.YP0104, SEQ ID NO: 5) was PCR amplified from Arabidopsis genomic DNA using primers 11 & 12 and was cloned in pMON77955 at BamH I-Stu I site resulting in a promoter construct pMON79359 which was used for transformation.

P-At.YP0104 without its native TATA box to TSS (SEQ ID NO: 6) was PCR amplified from Arabidopsis genomic DNA using primers 13 & 14 and was cloned in to Xho I site of pMON99667 to generate a promoter construct pMON79356, comprising the engineered P-At.YP0104 with the 35S TATA box to TSS (SEQ ID NO: 14) which was used for transformation.

P-At.YP0104 without its native TATA box to TSS (SEQ ID NO: 6) was PCR amplified from Arabidopsis genomic DNA using primers 13 & 14 and was cloned into Xho I site of pMON99668 to generate a promoter construct pMON79365, comprising the engineered P-At.YP0104 with the Os.Act1 TATA box to TSS (SEQ ID NO: 15) which was used for transformation.

The native Glycine max promoter GM.571 (P-Gm.700981571, aka P-Gm.571, SEQ ID NO: 7) full length was extracted from pMON57310 as a Not I-Nco I fragment, was end-filled using klenow and was cloned in to Stu I site of base vector pMON77955 resulting in a promoter construct pMON79361 which was used for transformation.

P-Gm.571 without its native TATA box to TSS (SEQ ID NO: 8) was PCR amplified from Arabidopsis genomic DNA using primers 15 & 16 and was cloned into Xho I site of pMON99667 to generate a promoter construct pMON79360, comprising the engineered P-Gm.571 with the 35S TATA box to TSS (SEQ ID NO: 16) which was used for transformation.

P-Gm.571 without its native TATA box to TSS (SEQ ID NO: 8) was PCR amplified from Arabidopsis genomic DNA using primers 15 & 16 and was cloned into Xho I site of pMON99668 to generate a promoter construct pMON79366, comprising the engineered P-Gm.571 with the Os.Act1 TATA box to TSS (SEQ ID NO: 17) which was used for transformation.

All regulatory elements were sub-cloned into a plant transformation vector operably linking the regulatory elements to the Zea mays HSP70 intron (described in U.S. Pat. No. 5,424,412, which is incorporated herein by reference in its entirety), the coding region for β-glucuronidase (GUS described in U.S. Pat. No. 5,599,670, which is incorporated herein by reference in its entirety), and the Agrobacterium tumefaciens NOS gene terminator, using methods commonly known to those skilled in the art.

### Example 2

### Plant Transformation and GUS Analysis

Corn plants were transformed with plant expression constructs for histochemical GUS analysis in plants. Plants were transformed using methods known to those skilled in the art. Particle bombardment of corn H99 immature zygotic embryos may be used to produce transgenic maize plants. Ears of maize H99 plants are collected 10-13 days after pollination from greenhouse grown plants and sterilized. Immature zygotic embryos of 1.2-1.5 mm are excised from the ear and incubated at 28° C. in the dark for 3-5 days before use as target tissue for bombardment. DNA comprising an isolated expression cassette containing either the full length or chimeric promoter, the selectable marker for kanamycin resistance (NPTII gene) and the screenable marker for β-D-Glucuronidase (GUS gene) is gel purified and used to coat 0.6 micron gold particles (Catalog #165-2262 Bio-Rad, Hercules, Calif.) for bombardment. Macro-carriers are loaded with the DNA-coated gold particles (Catalog #165-2335 Bio-Rad, Hercules Calif.). The embryos are transferred onto osmotic medium scutellum side up. A PDS 1000/He biolistic gun is used for transformation (Catalog #165-2257 Bio-Rad, Hercules Calif.). Bombarded immature embryos are cultured and transgenic calli are selected and transferred to shoot formation medium. Transgenic corn plants are regenerated from the transgenic calli and transferred to the greenhouse.

GUS activity is qualitatively and quantitatively measured using methods known to those skilled in the art. Plant tissue samples are collected from the same tissue for both the qualitative and quantitative assays. For qualitative analysis, whole tissue sections are incubated with the GUS staining solution X-Gluc (5-bromo-4-chloro-3-indolyl-β-glucuronide) (1 milligram/milliliter) for an appropriate length of time, rinsed, and visually inspected for blue coloration. For quantitative analysis, total protein is first extracted from each tissue sample. One microgram of total protein is used in a with the fluorogenic substrate 4-methyleumbelliferyl-β-D-glucuronide (MUG) in a total reaction volume of 50 μl (microliters). The reaction product 4-methlyumbelliferone (4-MU) is maximally fluorescent at high pH, where the hydroxyl group is ionized. Addition of a basic solution of sodium carbonate simultaneously stops the assay and adjusts the pH for quantifying the fluorescent product. Fluorescence is measured with excitation at 365 nm, emission at 445 nm using a Fluoromax-3 with Micromax Reader, with slit width set at excitation 2 nm and emission 3 nm. The GUS activity is expressed as pmole of 4-MU/micrograms of protein/hour (pMole of 4-MU/μg protein/hour).

### Example 3

### Promoter Analysis in Plants Subjected to Cold and Desiccation Stresses

Corn plants representing ten F1 events were transformed with each of the following constructs: pMON79353 (comprising SEQ ID NO: 1, P-At.rd29A), pMON79354 (comprising SEQ ID NO: 11, Chimeric P-At.rd29A/CaMV35S), pMON79367 (SEQ ID NO: 12, chimeric P-At.rd29A/Ract1), pMON79358 (SEQ ID NO: 3, P-At.GolS3), pMON79362 (SEQ ID NO: 13, Chimeric P-At.GolS3/CaMV35S), pMON79359 (SEQ ID NO: 5, P-At.YP0104), pMON79356 (SEQ ID NO: 14, Chimeric P-At.YP0104/CaMV35S), pMON79365 (SEQ ID NO: 15, Chimeric P-AtYP0104/Ract1), pMON79361 (SEQ ID NO: 7, P-Gm.571), pMON79360 (SEQ ID NO: 16, Chimeric P-Gm.571/CaMV35S), and pMON79366 (SEQ ID NO: 17, Chimeric P-Gm.571/Ract1). Each regulatory element was operably linked to the GUS coding region and the transformants analyzed for GUS activity as described above.

For cold stress experiments, roots of three-leaf stage plants (V3) were thoroughly washed in running water and then transferred into a test tube containing de-ionized water. The seedlings were subjected to cold stress by exposing to a temperature of 150 C for 24 h in presence of 800 micro mole/mt2/sec of light in a growth chamber. At the end of the stress treatment leaf and root tissues are sampled for both qualitative and quantitative GUS activity.

For water stress/desiccation experiments, water stress was imposed at three-leaf (V3) stage by withholding irrigation in the green house. Individual pots are weighed every day and it is monitored till it looses 50% of the initial weight. At this stage plants exhibited wilting symptom such as inward curling of leaf (V-shape). It takes about 4 to 5 days to reach this stage depending on environmental conditions. Leaf and root tissues were sampled for both qualitative and quantitative GUS activity once plants shows above-mentioned symptoms.

Mean levels of GUS activity (pMole of MU/ug protein/hour) for each stage of plant development and organ tested are provided as mean GUS activity+/−standard error (SE) measurements.

The full-length dicot promoters At.rd29A, Gm571, At.GolS3 and At.YP0104, when stably expressed in corn (monocot) did not exhibit GUS expression. However, when the native TATA box to transcription start site (TSS) was substituted with the TATA box to TSS region from either the CaMV35S promoter or the Rice Actin 1 promoter, significant expression was observed in the monocot plant.

The Arabidopsis (dicot) promoter At.Rd29A with its native TATA box to transcription start site (TSS) did not reveal any GUS expression in corn (a monocot). However, when the native TATA box to TSS was substituted with either the CaMV35S promoter TATA box to TSS region or the rice Actin 1 promoter TATA box to TSS region, both cold and desiccation induced expression was observed. Plants transformed with pMON79354 (At.rd29A/CaMV35S chimeric promoter) were compared to plants transformed with pMON79353 (dicot promoter At.rd29A). Results demonstrating cold- and desiccation-induced constitutive expression are shown in Table 2. Plants transformed with pMON79367 (At.rd29A/Rice Actin 1 chimeric promoter) were compared to plants transformed with pMON79353 (dicot promoter At.rd29A). Results showing cold- and desiccation-induced root and leaf expression are shown in Table 3.

TABLE 2
Expression of the Chimeric Promoter rd29A/CaMV35S (pMON79354)

| Stages | Organ | Inducer | Range | Mean ± SE |
|---|---|---|---|---|
| Imbibed seed | Embryo | — | 63.30-100.41 | 89.53 ± 3.49 |
| Imbibed seed | Endosperm | — | 12.81-59.19 | 34.01 ± 4.72 |
| 3 DAG | Root | Ctrl | 2.78-38.20 | 17.78 ± 5.22 |
| 3 DAG | Root | Cold | 2.41-56.22 | 26.02 ± 6.37 |
| V3 | Root | Ctrl | 10.14-10.14 | 10.14 ± 0.00 |
| V3 | Root | Cold | 71.38-135.85 | 103.73 ± 9.48 |
| V3 | Root | Des1 | 139.55-176.95 | 150.94 ± 6.87 |
| V3 | Root | Des2 | 74.10-145.65 | 110.88 ± 20.68 |
| V3 | Root | 1DAR | 64.14-152.81 | 101.89 ± 15.37 |
| V7 | Root | Ctrl | 53.32-123.09 | 80.19 ± 11.36 |
| V7 | Root | Des1 | 9.06-334.98 | 125.76 ± 54.93 |
| V7 | Root | Des2 | 192.87-306.17 | 259.25 ± 34.12 |
| V7 | Root | 1DAR | 105.89-295.34 | 213.50 ± 56.19 |
| VT | Root | Ctrl | 10.76-28.53 | 20.94 ± 5.29 |
| VT | Root | Des1 | <0.1-<0.1 | <0.1 ± 0.00 |
| VT | Root | Des2 | 29.28-83.37 | 53.33 ± 13.80 |
| VT | Root | 1DAR | <0.1-<0.1 | <0.1 ± 0.00 |
| 3 DAG | Coleoptile | Ctrl | 12.99-30.25 | 20.48 ± 2.95 |
| 3 DAG | Coleoptile | Cold | 9.04-47.94 | 25.85 ± 6.82 |
| V3 | Leaf | Ctrl | 4.91-7.12 | 5.84 ± 0.53 |
| V3 | Leaf | Cold | 39.88-120.94 | 69.93 ± 10.15 |
| V3 | Leaf | Des1 | 81.81-127.77 | 93.27 ± 8.68 |
| V3 | Leaf | Des2 | 6.52-188.97 | 94.46 ± 28.18 |
| V3 | Leaf | 1DAR | 55.27-144.63 | 98.15 ± 15.37 |
| V7 | Leaf - Mature | Ctrl | 15.23-53.02 | 35.51 ± 7.42 |
| V7 | Leaf - Mature | Des1 | 5.00-93.29 | 37.28 ± 19.77 |
| V7 | Leaf - Mature | Des2 | 14.35-20.14 | 16.59 ± 1.79 |
| V7 | Leaf - Mature | 1DAR | 11.18-18.61 | 14.18 ± 1.58 |
| VT | Leaf - Mature | Ctrl | 9.78-24.55 | 17.17 ± 7.38 |
| VT | Leaf - | Sens. | 34.51-44.76 | 39.63 ± 5.12 |
| | Senescence | | | |
| VT | Leaf - Mature | Des1 | 9.64-9.64 | 9.64 ± 0.00 |
| VT | Leaf - Mature | Des2 | 12.07-92.95 | 38.68 ± 14.71 |
| VT | Leaf - Mature | 1DAR | 9.69-24.52 | 14.71 ± 3.44 |
| VT | Cob | Ctrl | 56.05-78.08 | 66.32 ± 6.40 |
| VT | Cob | Des1 | 37.19-49.23 | 42.33 ± 3.59 |
| VT | Cob | Des2 | 34.69-151.23 | 92.68 ± 26.16 |
| VT | cob | 1DAR | 58.49-116.46 | 84.35 ± 12.46 |
| VT | Silk | Ctrl | 10.96-23.00 | 16.98 ± 6.02 |
| VT | Silk | Des1 | 21.76-39.49 | 30.38 ± 5.13 |
| VT | Silk | Des2 | 16.77-53.47 | 40.94 ± 6.72 |
| VT | Silk | 1DAR | 36.70-68.00 | 52.40 ± 6.86 |
| VT | Internode | — | 25.69-32.30 | 28.48 ± 1.98 |
| VT | Anther | — | 112.10-270.45 | 168.81 ± 35.37 |
| VT | Pollen | — | 86.22-313.57 | 157.38 ± 40.09 |
| 21 DAP | Embryo | — | 3.35-17.41 | 10.20 ± 1.98 |
| 35 DAP | Embryo | — | 2.34-90.22 | 36.54 ± 10.82 |
| 7 DAP | Kernel | — | 21.63-158.13 | 71.49 ± 7.47 |
| 21 DAP | Endosperm | — | 129.45-325.73 | 177.49 ± 11.62 |
| 35 DAP | Endosperm | — | 2.35-14.57 | 9.38 ± 1.18 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V 3 - three leaf stage;
V7 - Seven leaf stage;
nd—not determined
Des1 - desiccation1 (70 to 75% RWC);
Des2 - desiccation2 (55 to 60% RWC);
DAR—day after recovery;
RWC—relaive water content

TABLE 3
Expression of the Chimeric Promoter rd29A/Rice Actin 1

| Stages | Organ | Inducer | Range | Mean ± SE |
|---|---|---|---|---|
| Imbibed seed | Embryo | — | 0.00-0.00 | 0 |
| Imbibed seed | Endosperm | — | 0.00-0.00 | 0 |
| 3 DAG | Root | Ctrl | 0.00-0.00 | 0 |
| 3 DAG | Root | Cold | 0.00-0.00 | 0 |
| V3 | Root | Ctrl | 0.10-0.04 | 2.62 ± 0.89 |
| V3 | Root | Cold | 0.11-5.75 | 2.32 ± 0.76 |
| V3 | Root | Des1 | 0.23-4.65 | 2.27 ± 1.11 |
| V3 | Root | Des2 | 0.16-7.87 | 2.60 ± 1.03 |
| V3 | Root | 1DAR | 1.36-7.97 | 2.99 ± 0.37 |
| V7 | Root | Ctrl | 0.87-1.24 | 1.05 ± 0.19 |
| V7 | Root | Des1 | 0.00-0.00 | 0 |
| V7 | Root | Des2 | 0.00-0.00 | 0 |
| V7 | Root | 1DAR | 0.44-24.18 | 6.55 ± 2.07 |
| VT | Root | Ctrl | 0.00-0.00 | 0 |
| VT | Root | Des1 | 0.00-0.00 | 0 |
| VT | Root | Des2 | 0.00-0.00 | 0 |
| VT | Root | 1DAR | 0.00-0.00 | 0 |
| 3 DAG | Coleoptile | Ctrl | 0.00-0.00 | 0 |
| 3 DAG | Coleoptile | Cold | 0.00-0.00 | 0 |
| V3 | Leaf | Ctrl | 1.18-26.82 | 14.00 ± 12.82 |
| V3 | Leaf | Cold | 0.13-90.87 | 28.67 ± 12.80 |
| V3 | Leaf | Des1 | 0.25-67.52 | 20.39 ± 12.40 |
| V3 | Leaf | Des2 | 0.92-49.97 | 10.27 ± 4.91 |
| V3 | Leaf | 1DAR | 0.94-27.74 | 10.66 ± 2.31 |
| V7 | Leaf - Mature | Ctrl | 4.75-4.75 | 4.75 ± 0 |
| V7 | Leaf - Mature | Des1 | 0.00-0.00 | 0 |
| V7 | Leaf - Mature | Des2 | 0.78-10.97 | 5.88 ± 5.10 |
| V7 | Leaf - Mature | 1DAR | 0.13-5.21 | 2.58 ± 1.47 |
| VT | Leaf - Mature | Ctrl | 0.00-0.00 | 0 |
| VT | Leaf - | Sens. | 0.00-0.00 | 0 |
| | Senescence | | | |
| VT | Leaf - Mature | Des1 | 0.00-0.00 | 0 |
| VT | Leaf - Mature | Des2 | 0.00-0.00 | 0 |
| VT | Leaf - Mature | 1DAR | 0.00-0.00 | 0 |
| VT | Cob | Ctrl | 0.00-0.00 | 0 |
| VT | Cob | Des1 | 0.00-0.00 | 0 |
| VT | Cob | Des2 | 0.00-0.00 | 0 |
| VT | cob | 1DAR | 0.00-0.00 | 0 |
| VT | Silk | Ctrl | 0.00-0.00 | 0 |
| VT | Silk | Des1 | 0.00-0.00 | 0 |
| VT | Silk | Des2 | 0.00-0.00 | 0 |
| VT | Silk | 1DAR | 0.00-0.00 | 0 |
| VT | Internode | — | 0.00-0.00 | 0 |
| VT | Anther | — | 0.00-0.00 | 0 |
| VT | Pollen | — | 0.00-0.00 | 0 |
| 21 DAP | Embryo | — | 0.00-0.00 | 0 |
| 35 DAP | Embryo | — | 0.00-0.00 | 0 |
| 7 DAP | Kernel | — | 0.00-0.00 | 0 |
| 21 DAP | Endosperm | — | 0.00-0.00 | 0 |
| 35 DAP | Endosperm | — | 0.00-0.00 | 0 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V 3 - three leaf stage;
V7 - Seven leaf stage;
nd—not determined
Des1 - desiccation1 (70 to 75% RWC);
Des2 - desiccation2 (55 to 60% RWC);
DAR—day after recovery;
RWC—relaive water content

The Glycine max (dicot) promoter Gm.571 with its native TATA box to transcription start site (TSS) did not reveal any GUS expression in corn (a monocot). However, when the native TATA box to TSS was substituted with either the CaMV35S promoter TATA box to TSS region or the rice Actin 1 promoter TATA box to TSS region, both cold and desiccation induced expression was observed. Plants transformed with pMON79360 (Gm.571/CaMV35S chimeric promoter) were compared to plants transformed with pMON79361 (dicot promoter Gm.571). Results showing cold- and desiccation-induced constitutive expression are shown in Table 4. Plants transformed with pMON79366 (Gm.571/Rice Actin 1 chimeric promoter) were compared to plants transformed with pMON79361 (dicot promoter Gm.571). Results showing constitutive expression are shown in Table 5.

TABLE 4
Expression of the Chimeric Promoter Gm.571/CaMV35S

| Stages | Organ | Inducer | Range | Mean ± SE |
|---|---|---|---|---|
| Imbibed seed | Embryo | — | 63.30-100.41 | 89.53 ± 3.49 |
| Imbibed seed | Endosperm | — | 12.81-59.19 | 34.01 ± 4.72 |
| 3 DAG | Root | Ctrl | 2.78-38.20 | 17.78 ± 5.22 |
| 3 DAG | Root | Cold | 2.41-56.22 | 26.02 ± 6.37 |
| V3 | Root | Ctrl | 10.14-10.14 | 10.14 ± 0.00 |
| V3 | Root | Cold | 71.38-135.85 | 103.73 ± 9.48 |
| V3 | Root | Des1 | 139.55-176.95 | 150.94 ± 6.87 |
| V3 | Root | Des2 | 74.10-145.65 | 110.88 ± 20.68 |
| V3 | Root | 1DAR | 64.14-152.81 | 101.89 ± 15.37 |
| V7 | Root | Ctrl | 53.32-123.09 | 80.19 ± 11.36 |
| V7 | Root | Des1 | 9.06-334.98 | 125.76 ± 54.93 |
| V7 | Root | Des2 | 192.87-306.17 | 259.25 ± 34.12 |
| V7 | Root | 1DAR | 105.89-295.34 | 213.50 ± 56.19 |
| VT | Root | Ctrl | 10.76-28.53 | 20.94 ± 5.29 |
| VT | Root | Des1 | <0.1-<0.1 | <0.1 ± 0.00 |
| VT | Root | Des2 | 29.28-83.37 | 53.33 ± 13.80 |
| VT | Root | 1DAR | <0.1-<0.1 | <0.1 ± 0.00 |
| 3 DAG | Coleoptile | Ctrl | 12.99-30.25 | 20.48 ± 2.95 |
| 3 DAG | Coleoptile | Cold | 9.04-47.94 | 25.85 ± 6.82 |
| V3 | Leaf | Ctrl | 4.91-7.12 | 5.84 ± 0.53 |
| V3 | Leaf | Cold | 39.88-120.94 | 69.93 ± 10.15 |
| V3 | Leaf | Des1 | 81.81-127.77 | 93.27 ± 8.68 |
| V3 | Leaf | Des2 | 6.52-188.97 | 94.46 ± 28.18 |
| V3 | Leaf | 1DAR | 55.27-144.63 | 98.15 ± 15.37 |
| V7 | Leaf - Mature | Ctrl | 15.23-53.02 | 35.51 ± 7.42 |
| V7 | Leaf - Mature | Des1 | 5.00-93.29 | 37.28 ± 19.77 |
| V7 | Leaf - Mature | Des2 | 14.35-20.14 | 16.59 ± 1.79 |
| V7 | Leaf - Mature | 1DAR | 11.18-18.61 | 14.18 ± 1.58 |
| VT | Leaf - Mature | Ctrl | 9.78-24.55 | 17.17 ± 7.38 |
| VT | Leaf - | Sens. | 34.51-44.76 | 39.63 ± 5.12 |
| | Senescence | | | |
| VT | Leaf - Mature | Des1 | 9.64-9.64 | 9.64 ± 0.00 |
| VT | Leaf - Mature | Des2 | 12.07-92.95 | 38.68 ± 14.71 |
| VT | Leaf - Mature | 1DAR | 9.69-24.52 | 14.71 ± 3.44 |
| VT | Cob | Ctrl | 56.05-78.08 | 66.32 ± 6.40 |
| VT | Cob | Des1 | 37.19-49.23 | 42.33 ± 3.59 |
| VT | Cob | Des2 | 34.69-151.23 | 92.68 ± 26.16 |
| VT | cob | 1DAR | 58.49-116.46 | 84.35 ± 12.46 |
| VT | Silk | Ctrl | 10.96-23.00 | 16.98 ± 6.02 |
| VT | Silk | Des1 | 21.76-39.49 | 30.38 ± 5.13 |
| VT | Silk | Des2 | 16.77-53.47 | 40.94 ± 6.72 |
| VT | Silk | 1DAR | 36.70-68.00 | 52.40 ± 6.86 |
| VT | Internode | — | 25.69-32.30 | 28.48 ± 1.98 |
| VT | Anther | — | 112.10-270.45 | 168.81 ± 35.37 |
| VT | Pollen | — | 86.22-313.57 | 157.38 ± 40.09 |
| 21 DAP | Embryo | — | 3.35-17.41 | 10.20 ± 1.98 |
| 35 DAP | Embryo | — | 2.34-90.22 | 36.54 ± 10.82 |
| 7 DAP | Kernel | — | 21.63-158.13 | 71.49 ± 7.47 |
| 21 DAP | Endosperm | — | 129.45-325.73 | 177.49 ± 11.62 |
| 35 DAP | Endosperm | — | 2.35-14.57 | 9.38 ± 1.18 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V 3 - three leaf stage;
V7 - Seven leaf stage;
nd—not determined
Des1 - desiccation1 (70 to 75% RWC);
Des2 - desiccation2 (55 to 60% RWC);
DAR—day after recovery;
RWC—relaive water content

TABLE 5
Expression of the Chimeric Promoter Gm.571/Rice Actin 1

| Stages | Organ | Inducer | Range | Mean ± SE |
|---|---|---|---|---|
| V3 | Root | — | 1.26-11.17 | 4.28 ± 1.80 |
| V7 | Root | — | 1.90-29.50 | 16.18 ± 7.55 |
| V3 | Leaf | — | 5.95-61.27 | 24.58 ± 12.48 |
| V7 | Leaf - Mature | — | 1.25-23.01 | 9.52 ± 3.24 |
| VT | Leaf - Mature | — | 12.03-20.41 | 16.22 ± 4.19 |
| VT | Leaf - Senescence | — | 1.77-26.55 | 12.28 ± 5.94 |
| VT | Cob | — | 1.96-59.43 | 23.31 ± 10.09 |
| VT | Anther | — | 2.96-25.66 | 11.13 ± 2.11 |
| VT | Silk | — | 97.67 126.80 | 112.24 ± 14.57 |
| 21 DAP | Embryo | — | 19.05-60.89 | 40.71 ± 10.10 |
| 10 DAP | Kernel | — | 15.06-38.49 | 21.01 ± 1.94 |
| 21 DAP | Endosperm | — | 2.07-64.92 | 27.66 ± 9.09 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V3 - three leaf stage;
V7 - Seven leaf stage;
nd—not determined

The Arabidopsis (dicot) promoter At.GolS3 with its native TATA box to transcription start site (TSS) did not reveal any GUS expression in corn (a monocot). However, when the native TATA box to TSS was substituted with either the CaMV35S promoter TATA box to TSS region or the rice Actin 1 promoter TATA box to TSS region, basal expression as well as both cold and desiccation induced expression was observed. Plants transformed with pMON79362 (At.GolS3/CaMV35S chimeric promoter) were compared to plants transformed with pMON79358 (dicot promoter At.GolS3). Results are shown in Table 6.

TABLE 6
Expression of the Chimeric Promoter At.GolS3/CaMV35S

| Stages | Organ | Inducer | Range | Mean ± SE |
|---|---|---|---|---|
| Imbibed seed | Embryo | — | 63.30-100.41 | 89.53 ± 3.49 |
| Imbibed seed | Endosperm | — | 12.81-59.19 | 34.01 ± 4.72 |
| 3 DAG | Root | Ctrl | 2.78-38.20 | 17.78 ± 5.22 |
| 3 DAG | Root | Cold | 2.41-56.22 | 26.02 ± 6.37 |
| V3 | Root | Ctrl | 10.14-10.14 | 10.14 ± 0.00 |
| V3 | Root | Cold | 71.38-135.85 | 103.73 ± 9.48 |
| V3 | Root | Des1 | 139.55-176.95 | 150.94 ± 6.87 |
| V3 | Root | Des2 | 74.10-145.65 | 110.88 ± 20.68 |
| V3 | Root | 1DAR | 64.14-152.81 | 101.89 ± 15.37 |
| V7 | Root | Ctrl | 53.32-123.09 | 80.19 ± 11.36 |
| V7 | Root | Des1 | 9.06-334.98 | 125.76 ± 54.93 |
| V7 | Root | Des2 | 192.87-306.17 | 259.25 ± 34.12 |
| V7 | Root | 1DAR | 105.89-295.34 | 213.50 ± 56.19 |
| VT | Root | Ctrl | 10.76-28.53 | 20.94 ± 5.29 |
| VT | Root | Des1 | <0.1-<0.1 | <0.1 ± 0.00 |
| VT | Root | Des2 | 29.28-83.37 | 53.33 ± 13.80 |
| VT | Root | 1DAR | <0.1-<0.1 | <0.1 ± 0.00 |
| 3 DAG | Coleoptile | Ctrl | 12.99-30.25 | 20.48 ± 2.95 |
| 3 DAG | Coleoptile | Cold | 9.04-47.94 | 25.85 ± 6.82 |
| V3 | Leaf | Ctrl | 4.91-7.12 | 5.84 ± 0.53 |
| V3 | Leaf | Cold | 39.88-120.94 | 69.93 ± 10.15 |
| V3 | Leaf | Des1 | 81.81-127.77 | 93.27 ± 8.68 |
| V3 | Leaf | Des2 | 6.52-188.97 | 94.46 ± 28.18 |
| V3 | Leaf | 1DAR | 55.27-144.63 | 98.15 ± 15.37 |
| V7 | Leaf - Mature | Ctrl | 15.23-53.02 | 35.51 ± 7.42 |
| V7 | Leaf - Mature | Des1 | 5.00-93.29 | 37.28 ± 19.77 |
| V7 | Leaf - Mature | Des2 | 14.35-20.14 | 16.59 ± 1.79 |
| V7 | Leaf - Mature | 1DAR | 11.18-18.61 | 14.18 ± 1.58 |
| VT | Leaf - Mature | Ctrl | 9.78-24.55 | 17.17 ± 7.38 |
| VT | Leaf - | Sens. | 34.51-44.76 | 39.63 ± 5.12 |
| | Senescence | | | |
| VT | Leaf - Mature | Des1 | 9.64-9.64 | 9.64 ± 0.00 |
| VT | Leaf - Mature | Des2 | 12.07-92.95 | 38.68 ± 14.71 |
| VT | Leaf - Mature | 1DAR | 9.69-24.52 | 14.71 ± 3.44 |
| VT | Cob | Ctrl | 56.05-78.08 | 66.32 ± 6.40 |
| VT | Cob | Des1 | 37.19-49.23 | 42.33 ± 3.59 |
| VT | Cob | Des2 | 34.69-151.23 | 92.68 ± 26.16 |
| VT | cob | 1DAR | 58.49-116.46 | 84.35 ± 12.46 |
| VT | Silk | Ctrl | 10.96-23.00 | 16.98 ± 6.02 |
| VT | Silk | Des1 | 21.76-39.49 | 30.38 ± 5.13 |
| VT | Silk | Des2 | 16.77-53.47 | 40.94 ± 6.72 |
| VT | Silk | 1DAR | 36.70-68.00 | 52.40 ± 6.86 |
| VT | Internode | — | 25.69-32.30 | 28.48 ± 1.98 |
| VT | Anther | — | 112.10-270.45 | 168.81 ± 35.37 |
| VT | Pollen | — | 86.22-313.57 | 157.38 ± 40.09 |
| 21 DAP | Embryo | — | 3.35-17.41 | 10.20 ± 1.98 |
| 35 DAP | Embryo | — | 2.34-90.22 | 36.54 ± 10.82 |
| 7 DAP | Kernel | — | 21.63-158.13 | 71.49 ± 7.47 |
| 21 DAP | Endosperm | — | 129.45-325.73 | 177.49 ± 11.62 |
| 35 DAP | Endosperm | — | 2.35-14.57 | 9.38 ± 1.18 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V3 - three leaf stage;
V7 - Seven leaf stage;
nd—not determined
Des1 - desiccation1 (70 to 75% RWC);
Des2 - desiccation2 (55 to 60% RWC);
DAR—day after recovery;
RWC—relaive water content

The Arabidopsis (dicot) promoter At.YP0104 with its native TATA box to transcription start site (TSS) did not reveal any GUS expression in corn (a monocot). However, when the native TATA box to TSS was substituted with either the CaMV35S promoter TATA box to TSS region or the rice Actin 1 promoter TATA box to TSS region, GUS expression was observed in root and leaf at different stages tested. Plants transformed with pMON79356 (At.YP0104/CaMV35S chimeric promoter) were compared to plants transformed with pMON79359 (dicot promoter At.YP0104). Results are shown in Table 7. Plants transformed with pMON79365 (At.YP0104/Rice Actin 1 chimeric promoter) were compared to plants transformed with pMON79359 (dicot promoter At.YP0104). Very low expression was observed in root, leaf, anther and embryo in plants transformed with the At.YP0104/Rice Actin 1 chimeric promoter (Table 8).

TABLE 7
Expression of the Chimeric Promoter At.YP0104/CaMV35S

| Stages | Organ | Inducer | Range | Mean ± SE |
|---|---|---|---|---|
| Imbibed seed | Embryo | — | 63.30-100.41 | 89.53 ± 3.49 |
| Imbibed seed | Endosperm | — | 12.81-59.19 | 34.01 ± 4.72 |
| 3 DAG | Root | Ctrl | 2.78-38.20 | 17.78 ± 5.22 |
| 3 DAG | Root | Cold | 2.41-56.22 | 26.02 ± 6.37 |
| V3 | Root | Ctrl | 10.14-10.14 | 10.14 ± 0.00 |
| V3 | Root | Cold | 71.38-135.85 | 103.73 ± 9.48 |
| V3 | Root | Des1 | 139.55-176.95 | 150.94 ± 6.87 |
| V3 | Root | Des2 | 74.10-145.65 | 110.88 ± 20.68 |
| V3 | Root | 1DAR | 64.14-152.81 | 101.89 ± 15.37 |
| V7 | Root | Ctrl | 53.32-123.09 | 80.19 ± 11.36 |
| V7 | Root | Des1 | 9.06-334.98 | 125.76 ± 54.93 |
| V7 | Root | Des2 | 192.87-306.17 | 259.25 ± 34.12 |
| V7 | Root | 1DAR | 105.89-295.34 | 213.50 ± 56.19 |
| VT | Root | Ctrl | 10.76-28.53 | 20.94 ± 5.29 |
| VT | Root | Des1 | <0.1-<0.1 | <0.1 ± 0.00 |
| VT | Root | Des2 | 29.28-83.37 | 53.33 ± 13.80 |
| VT | Root | 1DAR | <0.1-<0.1 | <0.1 ± 0.00 |
| 3 DAG | Coleoptile | Ctrl | 12.99-30.25 | 20.48 ± 2.95 |
| 3 DAG | Coleoptile | Cold | 9.04-47.94 | 25.85 ± 6.82 |
| V3 | Leaf | Ctrl | 4.91-7.12 | 5.84 ± 0.53 |
| V3 | Leaf | Cold | 39.88-120.94 | 69.93 ± 10.15 |
| V3 | Leaf | Des1 | 81.81-127.77 | 93.27 ± 8.68 |
| V3 | Leaf | Des2 | 6.52-188.97 | 94.46 ± 28.18 |
| V3 | Leaf | 1DAR | 55.27-144.63 | 98.15 ± 15.37 |
| V7 | Leaf - Mature | Ctrl | 15.23-53.02 | 35.51 ± 7.42 |
| V7 | Leaf - Mature | Des1 | 5.00-93.29 | 37.28 ± 19.77 |
| V7 | Leaf - Mature | Des2 | 14.35-20.14 | 16.59 ± 1.79 |
| V7 | Leaf - Mature | 1DAR | 11.18-18.61 | 14.18 ± 1.58 |
| VT | Leaf - Mature | Ctrl | 9.78-24.55 | 17.17 ± 7.38 |
| VT | Leaf - | Sens. | 34.51-44.76 | 39.63 ± 5.12 |
| | Senescence | | | |
| VT | Leaf - Mature | Des1 | 9.64-9.64 | 9.64 ± 0.00 |
| VT | Leaf - Mature | Des2 | 12.07-92.95 | 38.68 ± 14.71 |
| VT | Leaf - Mature | 1DAR | 9.69-24.52 | 14.71 ± 3.44 |
| VT | Cob | Ctrl | 56.05-78.08 | 66.32 ± 6.40 |
| VT | Cob | Des1 | 37.19-49.23 | 42.33 ± 3.59 |
| VT | Cob | Des2 | 34.69-151.23 | 92.68 ± 26.16 |
| VT | cob | 1DAR | 58.49-116.46 | 84.35 ± 12.46 |
| VT | Silk | Ctrl | 10.96-23.00 | 16.98 ± 6.02 |
| VT | Silk | Des1 | 21.76-39.49 | 30.38 ± 5.13 |
| VT | Silk | Des2 | 16.77-53.47 | 40.94 ± 6.72 |
| VT | Silk | 1DAR | 36.70-68.00 | 52.40 ± 6.86 |
| VT | Internode | — | 25.69-32.30 | 28.48 ± 1.98 |
| VT | Anther | — | 112.10-270.45 | 168.81 ± 35.37 |
| VT | Pollen | — | 86.22-313.57 | 157.38 ± 40.09 |
| 21 DAP | Embryo | — | 3.35-17.41 | 10.20 ± 1.98 |
| 35 DAP | Embryo | — | 2.34-90.22 | 36.54 ± 10.82 |
| 7 DAP | Kernel | — | 21.63-158.13 | 71.49 ± 7.47 |
| 21 DAP | Endosperm | — | 129.45-325.73 | 177.49 ± 11.62 |
| 35 DAP | Endosperm | — | 2.35-14.57 | 9.38 ± 1.18 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V3 - three leaf stage;
V7 - Seven leaf stage;
nd—not determined
Des1 - desiccation1 (70 to 75% RWC);
Des2 - desiccation2 (55 to 60% RWC);
DAR—day after recovery;
RWC—relaive water content

TABLE 8
Expression of the Chimeric Promoter At.YP0104/Rice Actin 1

| Stages | Organ | Inducer | Range | Mean ± SE |
|---|---|---|---|---|
| V3 | Root | — | 1.88-1.88 | 1.88 ± 0 |
| V7 | Root | — | 0.00-0.00 | 0 |
| VT | Root | — | 0.00-0.00 | 0 |
| V3 | Leaf | — | 3.30-3.30 | 3.30 ± 0 |
| V7 | Leaf - Mature | — | 6.84-6.84 | 6.84 ± 0 |
| VT | Leaf - Mature | — | 0.00-0.00 | 0 |
| VT | Leaf - Senescence | — | 0.00-0.00 | 0 |
| VT | Cob | — | 0.00-0.00 | 0 |
| VT | Anther | — | 9.00-15.12 | 12.06 ± 3.06 |
| VT | Silk | — | 0.00 0.00 | 0 |
| 21 DAP | Embryo | — | 3.65-6.10 | 4.88 ± 1.22 |
| 10 DAP | Kernel | — | 0.00-0.00 | 0 |
| 21 DAP | Endosperm | — | 0.00-0.00 | 0 |

Range - lowest and highest activity of individual seedlings across events;
Mean/SE - overall mean across all the events
DAG—Days After Germination;
DAP—Days After Pollination;
Em—Embryo;
En—Endosperm;
VT - Tasseling stage;
IS—Imbibed seed;
C—coleoptile;
R—Root;
L—Leaf;
V3 - three leaf stage;
V7 - Seven leaf stage;
nd—not determined

The present invention thus provides polynucleotide constructs comprising regulatory elements that can modulate expression of an operably linked transcribable polynucleotide molecule and a transgenic plant stably transformed with the polynucleotide construct.

From the examples given, the present invention thus provides chimeric regulatory elements that are useful for modulating the expression of an operably linked transcribable polynucleotide molecule. In particular, the present invention includes and provides chimeric regulatory elements that allow dicot promoters to express in monocot plants. The present invention also provides a method for assembling polynucleotide constructs comprising the isolated regulatory elements and isolated promoter fragments, and for creating a transgenic plant stably transformed with the polynucleotide construct.

Having illustrated and described the principles of the present invention, it should be apparent to persons skilled in the art that the invention can be modified in arrangement and detail without departing from such principles. We claim all modifications that are within the spirit and scope of the appended claims. All patent documents cited in this specification are incorporated herein by reference to the same extent as if each individual was specifically and individually indicated to be incorporated by reference.

We claim:

1. A regulatory polynucleotide molecule wherein said polynucleotide molecule comprises a promoter from a dicotyledonous gene, or a complement thereof, wherein said promoter comprises a portion from the TATA box to the transcription start site, in which the native portion of the promoter from the TATA box to the transcription start site is substituted with the TATA box to the transcription start site portion of another promoter selected from the group consisting of a plant virus promoter and a promoter from a monocotyledonous gene.

2. The regulatory polynucleotide molecule of claim 1, wherein said promoter from said dicotyledonous gene is selected from the group consisting of AtRd29A, Gm571, At.GolS3 and AtYP0104.

3. The regulatory polynucleotide molecule of claim 2, in which said promoter from a monocotyledonous gene is the promoter from the Rice Actin 1 gene.

4. The regulatory polynucleotide molecule of claim 2, in which said plant virus promoter is the 35S promoter from CaMV.

5. The regulatory polynucleotide molecule of claim 1, wherein said polynucleotide molecule is selected from the group consisting of SEQ ID NO: 11 through SEQ ID NO: 17.

6. The regulatory polynucleotide molecule of claim 1 wherein said polynucleotide molecule comprises a nucleic acid sequence that hybridizes under high stringency conditions with a sequence selected from the group consisting of SEQ ID NO: 11 through SEQ ID NO: 17, or any complement thereof.

7. The regulatory polynucleotide molecule of claim 1 wherein said polynucleotide molecule, or any complement thereof, comprises a nucleic acid sequence wherein the nucleic acid sequence exhibits an 80% or greater identity to a sequence selected from the group consisting of SEQ ID NO: 11 through SEQ ID NO: 17.

8. The regulatory polynucleotide molecule of claim 1 wherein said polynucleotide molecule, or any complement thereof, comprises a nucleic acid sequence wherein the nucleic acid sequence exhibits a 90% or greater identity to a sequence selected from the group consisting of SEQ ID NO: 11 through SEQ ID NO: 17.

9. A polynucleotide construct comprising the regulatory polynucleotide molecule of claim 1, operably linked to a transcribable polynucleotide molecule.

10. The polynucleotide construct of claim 9, wherein said regulatory polynucleotide molecule comprises a nucleic acid sequence selected from the group consisting of SEQ ID NO: 11 through SEQ ID NO: 17.

11. The polynucleotide construct of claim 9, wherein said regulatory polynucleotide molecule, or any complement thereof, comprises a polynucleotide sequence which exhibits an 80% or greater identity to a sequence selected from the group consisting of SEQ ID NO: 11 through SEQ ID NO: 17.

12. A transgenic plant cell transformed with the polynucleotide construct of claim 9.

13. A transgenic plant transformed with the polynucleotide construct of claim 9.

14. A seed of said transgenic plant of claim 13.

15. A progeny of the transgenic plant of claim 13.

16. A method of improving the expression of a dicot promoter in a monocot plant comprising substituting the native portion of the promoter from the TATA box to the transcription start site with the TATA box to the transcription start site from a promoter selected from the group consisting of a plant virus promoter and a monocot promoter.
